(19) United States
(12) Patent Application Publication
(10) Pub. No.: US 2025/0281533 A1
(43) Pub. Date: Sep. 11, 2025

(54) POLYPEPTIDE SPECIFIC FOR MUCIN 1 AND USE THEREOF

(71) Applicant: LG CHEM, LTD., Seoul (KR)

(72) Inventors: Ji Hyun LEE, Daejeon (KR); Hae Youn LEE, Daejeon (KR); Yoon Aa CHOI, Daejeon (KR); Dae Gwan YI, Daejeon (KR); Jungwon CHOI, Daejeon (KR); Areum PARK, Daejeon (KR); Saem JUNG, Daejeon (KR); Eu Rim SONG, Daejeon (KR); Kyubong NA, Daejeon (KR); Min Jeong PARK, Daejeon (KR); Eun Ji JEUN, Daejeon (KR); Kyuhong CHOE, Daejeon (KR); Hyoju YI, Daejeon (KR); Hee Jung YANG, Daejeon (KR); Sung Woong JANG, Daejeon (KR)

(73) Assignee: LG CHEM, LTD., Seoul (KR)

(21) Appl. No.: 18/259,205

(22) PCT Filed: Dec. 24, 2021
(86) PCT No.: PCT/KR2021/019790

§ 371 Date: Jun. 23, 2023

(30) Foreign Application Priority Data
Dec. 24, 2020 (KR) 10-2020-0183768
Oct. 15, 2021 (KR) 10-2021-0137757

(51) Int. Cl. A61K 35/17; A61K 40/11; A61K 40/31; A61K 40/42; A61P 35/00; C07K 14/705; C07K 14/725; C07K 16/30; C12N 5/0783; C12N 15/86

(52) U.S. Cl. CPC A61K 35/17; A61K 40/11; A61K 40/31; A61K 40/4257; A61P 35/00; C07K 14/7051; C07K 14/70517; C07K 14/70521; C07K 16/3092; C12N 5/0636; C12N 15/86; A61K 2239/13; A61K 2239/17; A61K 2239/21; A61K 2239/54; C07K 2317/53; C07K 2317/565; C07K 2317/622; C07K 2319/02; C07K 2319/03; C12N 2510/00; C12N 2740/15043

(57) ABSTRACT

The present invention relates to a polypeptide binding to mucin 1, an isolated polynucleotide encoding same, a vector carrying the polynucleotide, and a cell including the vector. In addition, the present invention relates to a chimeric antigen receptor including the polypeptide binding to mucin 1, an isolated polynucleotide encoding the chimeric antigen receptor, a vector carrying the polynucleotide, an immune cell expressing the chimeric antigen receptor, a composition comprising same for treatment of cancer, and a method for treatment of cancer.

### TECHNICAL FIELD

### Cross-Reference to Related Application

This application is a 35 U.S.C. 371 National Phase Entry Application from PCT/KR2021/019790 filed on Dec. 24, 2021, which claims priority from, Korean Patent Application Nos. 10-2020-0183768 and 10-2021-0137757, filed on Dec. 24, 2020 and Oct. 15, 2021, respectively, the disclosures of which are hereby incorporated by reference herein in their entirety.

The present application includes a Sequence Listing filed in electronic format. The Sequence Listing is entitled “Corrected_OPP20230,750US_Sequence listing.txt” created on Aug. 6, 2024, and is 73,411 bytes in size. The information in the electronic format of the Sequence Listing is part of the present application and is incorporated herein by reference in its entirety.

### TECHNICAL FIELD

Provided is a polypeptide binding to mucin1, an isolated polynucleotide encoding the same, a vector comprising the polynucleotide, and a cell comprising the vector. Further, provided is a chimeric antigen receptor comprising the polypeptide binding to mucin1, an isolated polynucleotide encoding the chimeric antigen receptor, a vector comprising the polynucleotide, an immune cell expressing the chimeric antigen receptor, and a composition for and a method of treating cancer, each comprising the same.

### BACKGROUND

Mucin1 is a membrane-bound glycoprotein present in epithelial cells of almost all organs in the body. Mucin1 is a heterodimeric protein formed by a non-covalent interaction of an N-terminal subunit (MUC1-N) and a C-terminal subunit (MUC1-C). MUC1-C forms an extracellular domain, a transmembrane domain, and a cytoplasmic tail. MUC1-N repeatedly includes tandem repeats (TRs) containing 20 amino acids (HGVTSAPDTRPAPGSTAPPA), including glycosylation sites, and interacts with the extracellular domain of MUC1-C and continues to be linked with MUC1-C.

In normal epithelial cells, mucin1 is expressed at a low level, and its expression is limited to the apical membranes of the epithelium. However, mucin1 is known to be overexpressed in solid cancer such as breast cancer, lung cancer, colorectal cancer, skin cancer, thyroid cancer, gastric cancer, pancreatic cancer, kidney cancer, ovarian cancer, cervical cancer, etc., and known to be randomly expressed on the entire cell surface and abnormally glycosylated. Therefore, mucin1 is known to be useful as a target molecule for detecting cancer lesions or as a target molecule for treating cancer.

### BRIEF SUMMARY

### Technical Problem

Currently, there are many reports of anti-MUC1 antibodies for cancer treatment. However, anticancer therapy using antibodies is applicable only to cancer cells that have antigens specifically recognized by the antibodies, duration of the effect is short, and resistance often occurs during prolonged use thereof. Accordingly, there is a demand for developing a technology capable of continuously exerting strong anticancer effects on various cancers by activating the immune system in the body.

### Technical Solution

There is provided a polypeptide binding to mucin1.

An embodiment provides an isolated polynucleotide encoding the mucin1-binding polypeptide.

Other embodiment provides a vector comprising the polynucleotide encoding the mucin1-binding polypeptide.

Other embodiment provides a cell comprising the vector comprising the polynucleotide encoding the mucin1-binding polypeptide.

Other embodiment provides a chimeric antigen receptor comprising the mucin1-binding polypeptide.

Other embodiment provides an isolated polynucleotide encoding the chimeric antigen receptor.

Other embodiment provides a vector comprising the polynucleotide encoding the chimeric antigen receptor.

Other embodiment provides an immune cell expressing the chimeric antigen receptor, or comprising the polynucleotide encoding the chimeric antigen receptor.

Other embodiment provides a composition for treating cancer, the composition comprising the mucin1-binding polypeptide; the isolated polynucleotide encoding the mucin1-binding polypeptide; the vector comprising the polynucleotide encoding the mucin1-binding polypeptide; the cell comprising the polynucleotide encoding the mucin1-binding polypeptide; the chimeric antigen receptor comprising the mucin1-binding polypeptide; the isolated polynucleotide encoding the chimeric antigen receptor; the vector comprising the polynucleotide encoding the chimeric antigen receptor; or the immune cell comprising the polynucleotide encoding the chimeric antigen receptor, or expressing the chimeric antigen receptor.

### BRIEF DESCRIPTION OF DRAWINGS

FIG. 1 shows results of analyzing cytokine secretion for evaluating responses of CAR-T cells according to one exemplary embodiment of the present disclosure, which were prepared using donor 03- and donor 04-derived T cells, against MUC1-positive tumor cells (T47D, HEK293T-V1);

FIG. 2 shows results of analyzing cytokine secretion for evaluating responses of CAR-T cells according to one exemplary embodiment of the present disclosure, which were prepared using donor 06-derived T cells, against MUC1-positive tumor cells (T47D);

FIG. 3 shows results of analyzing cytokine secretion for evaluating responses of CAR-T cells according to one exemplary embodiment of the present disclosure, which were prepared using donor 06-derived T cells, against cells not expressing mucin1 (HEK293T);

FIG. 4 shows results of testing cytotoxicity of CAR-T cells according to one exemplary embodiment of the present disclosure, which were prepared using donor 03-derived T cells, against a human breast cancer cell line T47D;

FIG. 5 shows results of testing cytotoxicity of CAR-T cells, which were prepared using donor 04-derived T cells, against a human breast cancer cell line T47D;

FIG. 6 shows results of testing cytotoxicity of CAR-T cells, which were prepared using donor 03-derived T cells, against a human embryonic kidney cell HEK293T;

FIG. 7 shows results of testing cytotoxicity of CAR-T cells, which were prepared using donor 04-derived T cells, against a human embryonic kidney cell HEK293T;

FIG. 8 shows results of testing cytotoxicity of CAR-T cells, which were prepared using donor 04-derived T cells, against HEK293T-V1 obtained by expressing exogenous MUC1-C in a human embryonic kidney cell line;

FIG. 9 shows results of testing cytotoxicity of CAR-T cells according to one exemplary embodiment of the present disclosure, which were prepared using donor 05-derived T cells, against a human breast cancer cell line T47D;

FIG. 10 shows results of testing cytotoxicity of CAR-T cells according to one exemplary embodiment of the present disclosure, which were prepared using donor 06-derived T cells, against a human breast cancer cell line T47D;

FIG. 11 shows results of testing cytotoxicity of CAR-T cells according to one exemplary embodiment of the present disclosure, which were prepared using donor 07-derived T cells, against a human embryonic kidney cell line HEK293T;

FIG. 12 shows results of testing cytotoxicity of CAR-T cells according to one exemplary embodiment of the present disclosure, which were prepared using donor 08-derived T cells, against a human breast cancer cell line HCC70 (#2: 2447_2H08; #3: 2447_3A01; #4: 2447_3A08; #5: 2447_3A09; #6: 2447_3A12; #7: 2447_3B07; #8: 2447_3C08; #9: 2447_3H02; #10: 2447_3H08);

FIG. 13 shows results of differential cytotoxicity of CAR-T cells according to one exemplary embodiment of the present disclosure against a human breast cancer cell line (HCC70) and a normal human breast cell line (MCF10A) at an effector cell (E): target cell (T) ratio of 0.3:1 (#2: 2447_2H08);

FIG. 14 shows results of differential cytotoxicity of CAR-T cells according to one exemplary embodiment of the present disclosure against a human breast cancer cell line (HCC70) and a normal human breast cell line (MCF10A) at an effector cell (E): target cell (T) ratio of 0.1:1 (#2: 2447_2H08);

FIG. 15 shows results of examining tumor growth inhibition after injection of CAR-T cells according to one exemplary embodiment of the present disclosure into cancer animal models which had been injected with PANC1-v1 cells (PANC1 pancreatic cancer cells expressing MUC1);

FIG. 16 shows results of examining the number of human immune cells (hCD45+ cells) in blood 14 days and 28 days after injection of CAR-T cells according to one exemplary embodiment of the present disclosure into cancer animal models which had been injected with PANC1-v1 cells (PANC1 pancreatic cancer cells expressing MUC1);

FIG. 17 shows results of examining tumor growth inhibition after injection of two different doses of CAR-T cells according to one exemplary embodiment of the present disclosure into cancer animal models which had been injected with PANC1-v1 cells (PANC1 pancreatic cancer cells expressing MUC1);

FIG. 18 shows results of examining tumor growth inhibition after injection of CAR-T cells according to one exemplary embodiment of the present disclosure into cancer animal models which had been injected with HCC1954 cells (breast cancer cells naturally expressing MUC1); and

FIG. 19 shows results of examining the number of human immune cells (hCD45+ cells) in blood at regular time intervals after injection of CAR-T cells according to one exemplary embodiment of the present disclosure into cancer animal models which had been injected with HCC1954 cells (breast cancer cells naturally expressing MUC1).

### DETAILED DESCRIPTION

According to one aspect of the present invention, provided is a polypeptide binding to mucin1. In one preferred embodiment, the polypeptide binding to mucin1 of the present disclosure may be an antibody or an antigen-binding fragment thereof.

In one embodiment, provided is a polypeptide binding to mucin1, the polypeptide comprising a pair of a heavy chain variable (VH) region and a light chain variable (VL) region selected from the following VH and VL regions:
a VH region comprising complementarity-determining region (CDR) 1, 2, and 3 represented by amino acid sequences of SEQ ID NO: 1, SEQ ID NO: 2, and SEQ ID NO: 3, respectively, and a VL region comprising CDR 1, 2, and 3 represented by amino acid sequences of SEQ ID NO: 4, SEQ ID NO: 5, and SEQ ID NO: 6, respectively;
a VH region comprising CDR 1, 2, and 3 represented by amino acid sequences of SEQ ID NO: 11, SEQ ID NO: 12, and SEQ ID NO: 13, respectively, and a VL region comprising CDR 1, 2, and 3 represented by amino acid sequences of SEQ ID NO: 14, SEQ ID NO: 15, and SEQ ID NO: 16, respectively;
a VH region comprising CDR 1, 2, and 3 represented by amino acid sequences of SEQ ID NO: 21, SEQ ID NO: 22, and SEQ ID NO: 23, respectively, and a VL region comprising CDR 1, 2, and 3 represented by amino acid sequences of SEQ ID NO: 24, SEQ ID NO: 25, and SEQ ID NO: 26, respectively;
a VH region comprising CDR 1, 2, and 3 represented by amino acid sequences of SEQ ID NO: 31, SEQ ID NO: 32, and SEQ ID NO: 33, respectively, and a VL region comprising CDR 1, 2, and 3 represented by amino acid sequences of SEQ ID NO: 34, SEQ ID NO: 35, and SEQ ID NO: 36, respectively;
a VH region comprising CDR 1, 2, and 3 represented by amino acid sequences of SEQ ID NO: 41, SEQ ID NO: 42, and SEQ ID NO: 43, respectively, and a VL region comprising CDR 1, 2, and 3 represented by amino acid sequences of SEQ ID NO: 44, SEQ ID NO: 45, and SEQ ID NO: 46, respectively;
a VH region comprising CDR 1, 2, and 3 represented by amino acid sequences of SEQ ID NO: 51, SEQ ID NO: 52, and SEQ ID NO: 53, respectively, and a VL region comprising CDR 1, 2, and 3 represented by amino acid sequences of SEQ ID NO: 54, SEQ ID NO: 55, and SEQ ID NO: 56, respectively;
a VH region comprising CDR 1, 2, and 3 represented by amino acid sequences of SEQ ID NO: 61, SEQ ID NO: 62, and SEQ ID NO: 63, respectively, and a VL region comprising CDR 1, 2, and 3 represented by amino acid sequences of SEQ ID NO: 64, SEQ ID NO: 65, and SEQ ID NO: 66, respectively;
a VH region comprising CDR 1, 2, and 3 represented by amino acid sequences of SEQ ID NO: 71, SEQ ID NO: 72, and SEQ ID NO: 73, respectively, and a VL region comprising CDR 1, 2, and 3 represented by amino acid sequences of SEQ ID NO: 74, SEQ ID NO: 75, and SEQ ID NO: 76, respectively;
a VH region comprising CDR 1, 2, and 3 represented by amino acid sequences of SEQ ID NO: 81, SEQ ID NO: 82, and SEQ ID NO: 83, respectively, and a VL region comprising CDR 1, 2, and 3 represented by amino acid sequences of SEQ ID NO: 84, SEQ ID NO: 85, and SEQ ID NO: 86, respectively; and
a VH region comprising CDR 1, 2, and 3 represented by amino acid sequences of SEQ ID NO: 91, SEQ ID NO: 92, and SEQ ID NO: 93, respectively, and a VL region comprising CDR 1, 2, and 3 represented by amino acid sequences of SEQ ID NO: 94, SEQ ID NO: 95, and SEQ ID NO: 96, respectively.

TABLE 1

| | CDR | | | | | |
|---|---|---|---|---|---|---|
| Clone | VH | | | VL | | |
| name | CDR1 | CDR2 | CDR3 | CDR1 | CDR2 | CDR3 |
| 2445_ | GFTVSSN | IYSGGST | DRGWNHG | SSEVGSR | KND | AAWDDSL |
| 1F09 | Y | (SEQ ID | MDV | Y | (SEQ ID | NGYV |
| | (SEQ ID | NO: 2) | (SEQ ID NO: | (SEQ ID | NO: 5) | (SEQ ID |
| | NO: 1) | | 3) | NO: 4) | | NO: 6) |
| 2447_ | GYTFTNY | ISAYNGNT | DPHILTGY | SLRTSY | GKT | HSRDSND |
| 2H08 | G | (SEQ ID | YRGGWFD | (SEQ ID | (SEQ ID | NYLEVV |
| | (SEQ ID | NO: 12) | P | NO: 14) | NO: 15) | (SEQ ID |
| | NO: 11) | | (SEQ ID NO: | | | NO: 16) |
| | | | 13) | | | |
| 2447_ | GFTFDDY | ISWNSGSI | DISSGWYP | SLRSYY | GKN | SSRDSSDD |
| 3A01 | A | (SEQ ID | GTFDY | (SEQ ID | (SEQ ID | VV |
| | (SEQ ID | NO: 22) | (SEQ ID NO: | NO: 24) | NO: 25) | (SEQ ID |
| | NO: 21) | | 23 | | | NO: 26) |
| 2447_ | GYTFTSY | ISAYNGNT | DRATIFGV | SIRSYS | GKN | NSRDSSG |
| 3A08 | G | (SEQ ID | VTPFDY | (SEQ ID | (SEQ ID | NRVV |
| | (SEQ ID | NO: 32) | (SEQ ID NO: | NO: 34) | NO: 35) | (SEQ ID |
| | NO: 31) | | 33 | | | NO: 36) |
| 2447_ | GFTFDDY | ISWNSGSI | DVSSGWY | SLRSYY | GKN | NSRDSGG |
| 3A09 | A | (SEQ ID | WYAFDI | (SEQ ID | (SEQ ID | SVV |
| | (SEQ ID | NO: 42) | (SEQ ID NO: | NO: 44) | NO: 45) | (SEQ ID |
| | NO: 41) | | 43) | | | NO: 46) |
| 2447_ | GYTFTSY | ISAYNGNT | DPHILTGY | SLRSYY | GKN | NSRDSSG |
| 3A12 | G | (SEQ ID | YRGGWFD | (SEQ ID | (SEQ ID | NHRV |
| | (SEQ ID | NO: 52) | P | NO: 54) | NO: 55) | (SEQ ID |
| | NO: 51) | | (SEQ ID NO: | | | NO: 56) |
| | | | 53) | | | |
| 2447_ | GFTFDDY | TSWDGGS | DHSSGWY | SLRSYY | GKN | NSRDSSG |
| 3B07 | A | T | NGGMDV | (SEQ ID | (SEQ ID | NHVV |
| | (SEQ ID | (SEQ ID | (SEQ ID NO: | NO: 64) | NO: 65) | (SEQ ID |
| | NO: 61) | NO: 62) | 63) | | | NO: 66) |
| 2447_ | GFTFDDY | ISWNSGSI | DRGSGYE | SLRSYY | GKN | NSRDSSG |
| 3C08 | A | (SEQ ID | GNYYGMD | (SEQ ID | (SEQ ID | NHYV |
| | (SEQ ID | NO: 72) | V | NO: 74) | NO: 75) | (SEQ ID |
| | NO: 71) | | (SEQ ID NO: | | | NO: 76) |
| | | | 73) | | | |
| 2447_ | GFTFDDY | ISWDSGSI | DVSSGWY | ILRSYY | GKN | NSRDSSG |
| 3H02 | A | (SEQ ID | WYAFDI | (SEQ ID | (SEQ ID | NRVV |
| | (SEQ ID | NO: 82) | (SEQ ID NO: | NO: 84) | NO: 85) | (SEQ ID |
| | NO: 81) | | 83) | | | NO: 86) |
| 2447_ | GFTFDDY | ISWNSGSI | DRSSGWY | SLRSYY | GKN | QSRDSSD |
| 3H08 | A | (SEQ ID | TGSFDY | (SEQ ID | (SEQ ID | NRVL |
| | (SEQ ID | NO: 92) | (SEQ ID NO: | NO: 94) | NO: 95) | (SEQ ID |
| | NO: 91) | | 93) | | | NO: 96) |

In another embodiment, provided is a polypeptide binding to mucin1, the polypeptide comprising a pair of a heavy chain variable (VH) region and a light chain variable (VL) region selected from the following VH and VL regions:
a VH region comprising an amino acid sequence of SEQ ID NO: 7 and a VL region comprising an amino acid sequence of SEQ ID NO: 8;
a VH region comprising an amino acid sequence of SEQ ID NO: 17 and a VL region comprising an amino acid sequence of SEQ ID NO: 18;
a VH region comprising an amino acid sequence of SEQ ID NO: 27 and a VL region comprising an amino acid sequence of SEQ ID NO: 28;
a VH region comprising an amino acid sequence of SEQ ID NO: 37 and a VL region comprising an amino acid sequence of SEQ ID NO: 38;
a VH region comprising an amino acid sequence of SEQ ID NO: 47 and a VL region comprising an amino acid sequence of SEQ ID NO: 48;
a VH region comprising an amino acid sequence of SEQ ID NO: 57 and a VL region comprising an amino acid sequence of SEQ ID NO: 58;
a VH region comprising an amino acid sequence of SEQ ID NO: 67 and a VL region comprising an amino acid sequence of SEQ ID NO: 68;
a VH region comprising an amino acid sequence of SEQ ID NO: 77 and a VL region comprising an amino acid sequence of SEQ ID NO: 78;
a VH region comprising an amino acid sequence of SEQ ID NO: 87 and a VL region comprising an amino acid sequence of SEQ ID NO: 88; and
a VH region comprising an amino acid sequence of SEQ ID NO: 97 and a VL region comprising an amino acid sequence of SEQ ID NO: 98.

TABLE 2

| Clone | | |
|---|---|---|
| name | VH | VL |
| 2445_ | EVQLLETGGGLIQPGGSLRLSCAAS | QSGLTQPPSASGTPGQRVTISCSGG |
| 1F09 | GFTVSSNYMSWVRQAPGKGLEWVS | SSEVGSRYVSWYQQLPGTAPRLLI |
| | VIYSGGSTYYADSVKGRFTISRDNS | YKNDRRPSGVPDRESGSKSGSSAS |
| | KNTLYLQMNSLRAEDTAVYYCARD | LAISGLRSEDEADYYCAAWDDSLN |
| | RGWNHGMDVWGQGTLVTVSS | GYVFGTGTKLTVL (SEQ ID NO: 8) |
| | (SEQ ID NO: 7) | |
| 2447_ | EVQLVQSGAEVKKPGASVKVSCKA | SYELTQDPAVSVALGQTVTITCQG |
| 2H08 | SGYTFTNYGISWVRQAPGQGLEWM | DSLRTSYAGWLQQKPGQAPVLVL |
| | GWISAYNGNTNYAQKLQGRVTMTT | YGKTSRPSGIPDRESGSTSGNTASL |
| | DTSTSTAYMELRSLRSDDTAVYYCA | TITGAQAEDEAEYFCHSRDSNDNY |
| | RDPHILTGYYRGGWFDPWGQGTTV | LEVVFGGGTKLTVL (SEQ ID NO: |
| | TVSS (SEQ ID NO: 17) | 18) |
| 2447_ | EVQLLESGGGLVQPGGSLRLSCAAS | SYELTQDPAVSVALGQTVRITCQG |
| 3A01 | GFTFDDYAMHWVRQAPGKGLEWV | DSLRSYYASWYQQKPGQAPVLVIY |
| | SGISWNSGSIVYADFVKGRFTISRDN | GKNNRPSGIPDRFSGSSSGNTASLT |
| | AKNSLYLQMNSLRAEDTALYYCAK | VTGAQAEDEADYYCSSRDSSDDV |
| | DISSGWYPGTFDYWGQGTLVTVSS | VFGGGTQLTVL (SEQ ID NO: 28) |
| | (SEQ ID NO: 27) | |
| 2447_ | EVQLVQSGAEVKKPGASVKVSCKA | SYELTQDPAVSVALGQTVRITCQG |
| 3A08 | SGYTFTSYGISWVRQAPGQGLEWM | DSIRSYSASWYQQKPGQAPRLVIY |
| | GWISAYNGNTNYAQKLQGRVTMTT | GKNNRPSGIPDRVSGSTSGNTASLT |
| | DTSTSTAYMELRSLRSDDTAVYYCA | VTGAQAEDEADYYCNSRDSSGNR |
| | RDRATIFGVVTPFDYWGQGTLVTVS | VVFGGGTQLTVL (SEQ ID NO: 38) |
| | S (SEQ ID NO: 37) | |
| 2447_ | EVQLLESGGGLVQPGRSLRLSCAAT | SYELTQDPAVSVALGQTVRITCQG |
| 3A09 | GFTFDDYAMHWVRQAPGKGLEWV | DSLRSYYASWYQQKPGQAPVLVIY |
| | SGISWNSGSIGYADSVKGRFTISRDN | GKNNRPSGIPGRFSGSSSGNTASLT |
| | AKNSLYLQMNSLRAEDTASYYCAK | VTGAQAEDEADYYCNSRDSGGSV |
| | DVSSGWYWYAFDIWGQGTLVTVSS | VFGGGTKLTVL (SEQ ID NO: 48) |
| | (SEQ ID NO: 47) | |
| 2447_ | EVQLVQSGAEVKKPGASVKVSCKA | SYELTQDPAVSVVLGQTVRITCQG |
| 3A12 | SGYTFTSYGISWVRQAPGQGLEWM | DSLRSYYASWYQQKPGQAPVLVIY |
| | GWISAYNGNTNYAQKLRGRVTMTT | GKNNRPSGIPDRESGSSSGNTASLTI |
| | DTSTSTAYMELRSLRSDDTAVYYCA | TGAQAEDEADYYCNSRDSSGNHR |
| | RDPHILTGYYRGGWFDPWGQGTLV | VFGGGTKLTVL (SEQ ID NO: 58) |
| | TVSS (SEQ ID NO: 57) | |
| 2447_ | QVQLVESGGVVVQPGGSLRLSCAA | SYELTQDPAVSVALGQTVRITCQG |
| 3B07 | SGFTFDDYAMHWVRQAPGRGLEW | DSLRSYYASWYQQKPGQAPVLVIY |
| | VSLTSWDGGSTYYADSVKGRFTISR | GKNNRPSGIPDRFSGSSSGNTASLTI |
| | DNSKNSLYLQMNSLRAEDTALYYC | TGAQAEDEADYYCNSRDSSGNHV |
| | AKDHSSGWYNGGMDVWGQGTMV | VFGGGTKLTVL (SEQ ID NO: 68) |
| | TVSS (SEQ ID NO: 67) | |
| 2447_ | EVQLLESGGGLVQPGGSLRLSCAAS | SYELTQDPAVSVALGQTVRITCQG |
| 3C08 | GFTFDDYAMHWVRQAPGKGLEWV | DSLRSYYASWYQQKPGQAPVLVIY |
| | SGISWNSGSIGYADSVKGRFTISRDN | GKNNRPSGIPDRFSGTTSGNTASLT |
| | AKNSLYLQMNSLRAEDTALYYCAK | ITGAQAEDEADYYCNSRDSSGNHY |
| | DRGSGYEGNYYGMDVWGQGTLVT | VFGTGTKVTVL (SEQ ID NO: 78) |
| | VSS (SEQ ID NO: 77) | |
| 2447_ | EVQLLESGGGLVQPGRSLRLSCAAS | SYELTQDPAVSVALGQTVRITCQG |
| 3H02 | GFTFDDYAMHWVRQAPGKGLEWV | DILRSYYASWYQQKPGQAPVLVIY |
| | SGISWDSGSIGYADSVKGRFTISRDN | GKNNRPSGIPDRFSGSSSGNTASLTI |
| | AKNSLYLQMNSLRAEDTALYYCAK | TGAQAEDEADYYCNSRDSSGNRV |
| | DVSSGWYWYAFDIWGQGTLVTVSS | VFGGGTKLTVL (SEQ ID NO: 88) |
| | (SEQ ID NO: 87) | |
| 2447_ | EVQLLESGGGLVQPGRSLRLSCAAS | SYELTQDPAVSVALGQTVRITCQG |
| 3H08 | GFTFDDYAMHWVRQAPGKGLEWV | DSLRSYYASWYRQKPGQAPVLVIY |
| | SGISWNSGSIDYADSVKGRFTISRDN | GKNNRPSGIPDRFSGSSSGNTASLTI |
| | SKNTLYLQMNSLRAEDTAVYYCAR | IGAQAEDEADYYCQSRDSSDNRVL |
| | DRSSGWYTGSFDYWGQGTLVTVSS | FGGGTKVTVL (SEQ ID NO: 98) |
| | (SEQ ID NO: 97) | |

As used herein, the term “antibody” collectively refers to a protein that specifically binds to a specific antigen, and is used in the broadest sense, and it may be a protein produced in the immune system by stimulation of an antigen or a protein synthesized chemically or prepared recombinantly. The type thereof is not particularly limited. Specifically, the antibody encompasses monoclonal antibodies (including full-length monoclonal antibodies), polyclonal antibodies, multispecific antibodies (e.g., bispecific antibodies), synthetic antibodies (also called antibody mimetics), chimeric antibodies, humanized antibodies, human antibodies, or antibody fusion proteins (also called antibody conjugates), provided such antibodies exhibit a desired biological activity.

An intact antibody (e.g., IgG-type) has a structure having two full-length light chains and two full-length heavy chains, each light chain associated with the heavy chain through disulfide bonds. A constant region of an antibody is divided into a heavy chain constant region and a light chain constant region, wherein the heavy chain constant region has a gamma (γ), mu (μ), alpha (α), delta (δ), or epsilon (ε) type, and a subclass of gamma1 (γ1), gamma2 (γ2), gamma3 (γ3), gamma4 (γ4), alpha1 (α1), or alpha2 (α2), and the light chain constant region has a kappa (κ) or lambda (λ) type.

The term “antigen-binding fragment” refers to a fragment of an antibody, which is able to specifically bind to an antigen even though at least part of amino acids present in the full-length chain is absent. Such a fragment is biologically active in that it binds to a target antigen, and competes with other antigen-binding molecules including an intact antibody for binding to a given epitope. The antigen-binding fragment may not include a constant heavy chain domain (i.e., CH2, CH3, and CH4 according to antibody isotypes) of Fc region of an intact antibody. Examples of the antigen-binding fragment may include single chain variable fragments (scFvs) (e.g., scFv, (scFv)₂, etc.), fragment antigen binding (Fab) (e.g., Fab, Fab′, F(ab′)₂, etc.), domain antibodies, peptibodies, minibodies, intrabodies, diabodies, triabodies, tetrabodies, or and single-chain antibodies, etc., but are not limited thereto. Further, the antigen-binding fragment may be scFv, or a fusion polypeptide (scFv-Fc), in which scFv is fused with an Fc region of an immunoglobulin (e.g., IgA, IgD, IgE, IgG (IgG1, IgG2, IgG3, IgG4), IgM, etc.), or a fusion polypeptide (scFv-Ck (kappa constant region) or scFv-Cλ (lambda constant region), in which scFv is fused with a light chain constant region (e.g., kappa or lambda), but is not limited thereto.

The term “heavy chain” is interpreted as having a meaning including a full-length heavy chain including a variable region domain VH including an amino acid sequence having a variable region sequence sufficient to impart specificity to an antigen and three constant region domains, C_{H1}, C_{H2} and C_{H3}, and a hinge, and fragments thereof. Further, the term “light chain” is interpreted as having a meaning including a full-length light chain including a variable region domain VL including an amino acid sequence having a variable region sequence sufficient to impart specificity to an antigen and a constant region domain CL, and fragments thereof.

The term “complementarity-determining region (CDR)” refers to a region in a variable region of an antibody, which imparts binding specificity or binding affinity to an antigen. Generally, there are three CDRs (CDR-H1, CDR-H2, CDR-H3) in a heavy chain variable region, and three CDRs (CDR-L1, CDR-L2, CDR-L3) in a light chain variable region. The CDRs may provide key contact residues for an antibody or a fragment thereof binding to an antigen or an epitope. The term “framework region (FR)” refers to non-CDR regions of variable regions of heavy and light chains. Generally, there are four FRs (FR-H1, FR-H2, FR-H3, and FR-H4) in a heavy chain variable region, and four FRs (FR-L1, FR-L2, FR-L3 and FR-L4) in a light chain variable region. The precise amino acid sequence boundaries of a given CDR or FR may be readily determined using any of a number of well-known schemes, such as Kabat numbering scheme, Chothia numbering scheme, Contact numbering scheme, IMGT numbering scheme, Aho numbering scheme, AbM numbering scheme, etc.

The term “variable region” refers to a domain of a heavy chain or light chain of an antibody, which is involved in binding of the antibody to an antigen. The heavy chain variable (VH) region and the light chain variable (VL) region generally have the similar structure, and each domain includes four conserved framework regions (FRs) and three CDRs.

In a specific embodiment, the mucin1-binding polypeptide of the present disclosure may be a single chain variable fragment (scFv), a peptibody, a fragment antigen binding (Fab), a monoclonal antibody, a bispecific antibody, a minibody, a domain antibody, a synthetic antibody, a chimeric antibody, a humanized antibody, a human antibody, or an antibody fusion protein, but is not limited thereto.

In a preferred specific embodiment, the mucin1-binding polypeptide of the present disclosure may be a single chain variable fragment (scFv).

The term “single chain variable fragment (scFv)” refers to a single chain antibody fragment, in which a heavy chain variable (VH) region and a light chain variable (VL) region of an antibody are linked via a covalent bond. The VH region and the VL region may be linked to each other directly or via a linker.

The linker may be a water-soluble and/or flexible linker. For example, the N-terminus of VH and the C-terminus of VL or the C-terminus of VH and the N-terminus of VL may be linked to each other via the linker. The linker may be a peptide linker, for example, having a length of 10 to 30 amino acids, 10 to 25 amino acids, 10 to 20 amino acids, 10 to 15 amino acids, 15 to 30 amino acids, 15 to 25 amino acids, 15 to 20 amino acids, specifically, 10, 11, 12, 13, 14, 15, 16, 17, 18, 19, 20, 21, 22, 23, 24, 25, 26, 27, 28, 29, or 30 amino acids, but is not limited thereto.

Amino acid sequences suitable for the linker are known in the art. For example, the linker may be rich in glycine for flexibility, and may include serine (Ser), asparagine (Asn), alanine (Ala), or threonine (Thr) for solubility. The linker may consist of a total of 1 to 100, 2 to 50, or 5 to 25 of one or more selected from the group consisting of Gly, Asn, Ser, Thr, and Ala. For non-limiting example, the amino acid sequence of the linker may be a sequence having various numbers of repeats of GGGS or GGGGS, for example, 2, 3, 4, and 5 repeats thereof. For example, the linker may be GGGGSGGGGSGGGGS, GGGGSGGGGSGGGGSGGGGS, or GGGGSGGGGSGGGAS, but is not limited thereto.

scFv retains antigen specificity of the original antibody despite removal of constant regions and introduction of a linker. scFv may be expressed from polynucleotides encoding the VH region and the VL region, but is not limited thereto.

In a preferred embodiment, provided is an scFv polypeptide comprising an amino acid sequence selected from the group consisting of SEQ ID NO: 9, SEQ ID NO: 19, SEQ ID NO: 29, SEQ ID NO: 39, SEQ ID NO: 49, SEQ ID NO: 59, SEQ ID NO: 69, SEQ ID NO: 79, SEQ ID NO: 89, and SEQ ID NO: 99.

In the present disclosure, variants retaining an activity equivalent to that of the antibody or the antigen-binding fragment, for example, variants having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% or more identity to the amino acid sequences described herein and retaining a biological activity equivalent thereto may be also included in the scope of the present disclosure.

According to another aspect of the present invention, provided is an isolated polynucleotide encoding the mucin1-binding polypeptide.

In one preferred embodiment, the polynucleotide may include a nucleotide sequence selected from the group consisting of SEQ ID NO: 10, SEQ ID NO: 20, SEQ ID NO: 30, SEQ ID NO: 40, SEQ ID NO: 50, SEQ ID NO: 60, SEQ ID NO: 70, SEQ ID NO: 80, SEQ ID NO: 90, and SEQ ID NO: 100. Further, the present invention may include an isolated polynucleotide encoding a polypeptide having about 90%, about 91%, about 92%, about 93%, about 94%, about 95%, about 96%, about 97%, about 98% or about 99% or more identity to the sequence and retaining equivalent activity thereto. In an embodiment, the polynucleotide may be codon-optimized for expression in humans.

According to still another aspect, provided is a vector comprising the polynucleotide encoding the mucin1-binding polypeptide.

In the vector, the polynucleotide may be operably linked to a promoter. The term “operatively linked” refers to a functional linkage between a nucleotide expression control sequence (e.g., a promoter sequence) and another nucleotide sequence. The control sequence may be operably linked to control transcription and/or translation of another nucleotide sequence.

The vector may be constructed as a vector for typically cloning or expression. The vector for expression may be those commonly used in the art to express a foreign protein in plants, animals, or microorganisms. The vector may be constructed through various methods known in the art.

The vector may be constructed using a prokaryotic cell or a eukaryotic cell as a host. For example, when the vector to be used is an expression vector and a prokaryotic cell is used as a host, the vector may generally include a strong promoter capable of initiating transcription (e.g., pL^{λ} promoter, CMV promoter, trp promoter, lac promoter, tac promoter, T7 promoter, etc.), a ribosome binding site for initiating translation, and a transcription/translation termination sequence. When a eukaryotic cell is used as a host, an origin of replication which is included in the vector and acts in the eukaryotic cell may include f1 origin of replication, SV40 origin of replication, pMB1 origin of replication, adeno origin of replication, AAV origin of replication, BBV origin of replication, etc., but is not limited thereto. Further, a promoter derived from genomes of mammalian cells (e.g., a metallothionein promoter) or a promoter derived from mammalian viruses (e.g., an adenovirus late promoter, a vaccinia virus 7.5K promoter, a SV40 promoter, a cytomegalovirus promoter, and a tk promoter of HSV) may be used, and a transcription termination sequence may be, in general, a polyadenylation sequence. As needed, the vector may further include an enhancer sequence, 5′- and 3′-untranslated regions, a secretion signal sequence, a splice junction, a selection marker (e.g., an antibiotic resistance gene), etc.

According to still another aspect, provided is a cell comprising the vector comprising the polynucleotide encoding the mucin1-binding polypeptide.

The cell may be obtained by introducing the vector into an appropriate host cell. The host cell comprising the vector may be useful for cloning or expressing the polynucleotide included in the vector. The host cell is a cell capable of stably and continuously cloning or expressing the vector, and any host cell known in the art may be used. For example, a prokaryotic cell may include, e.g., E. coli JM109, E. coli BL21, E. coli RR1, E. coli LE392, E. coli B, E. coli X 1776, E. coli W3110, strains of the genus Bacillus such as Bacillus subtilis, Bacillus thuringiensis, and strains of the family enterobacteriaceae such as Salmonella typhimurium, Serratia marcescens and various Pseudomonas species. When transformed into a eukaryotic cell, the host cell may include a yeast (Saccharomyces cerevisiae) cell, an insect cell, a plant cell, and an animal cell, e.g., Sp2/0, Chinese hamster ovary (CHO) K1, CHO DG44, PER.C6, W138, BHK, COS-7, 293, HepG2, Huh7, 3T3, RIN, MDCK cell line, etc., but is not limited thereto.

Delivery (introduction) of the polynucleotide or the vector comprising the same into the host cell may be carried out by using a delivering method widely known in the art. With regard to the delivering method, for example, when the host cell is a prokaryotic cell, a CaCl₂) method or an electroporation method may be used, and when the host cell is a eukaryotic cell, a microinjection method, a calcium phosphate precipitation method, an electroporation method, a liposome-mediated transfection method, and gene bombardment may be used, but is not limited thereto.

A method of selecting the transformed host cell may be easily carried out using a phenotype expressed by a selection marker according to a method well known in the art. For example, when the selection marker is a specific antibiotic resistance gene, the transformant may be easily selected by culturing the transformant in a medium containing the antibiotic. The transformed host cells may be cultured for a period sufficient for polypeptide expression or secretion, and the polypeptide may be separated and purified by a method of separating and purifying proteins commonly used, for example, a method of using solubility, such as salting out, solvent precipitation, etc., a method of using a molecular weight difference, such as dialysis, ultrafiltration, gel filtration, sodium dodecyl sulfate-polyacrylamide gel electrophoresis, etc., a method of using a charge, such as ion exchange chromatography or hydroxylapatite chromatography, etc., a method of using specific affinity, such as affinity chromatography, etc., a method of using a hydrophobicity difference, such as reverse-phase high performance liquid chromatography, etc., a method of using an isoelectric point difference, such as isoelectric point electrophoresis, etc.

According to still another aspect of the present invention, provided is a chimeric antigen receptor (CAR) comprising the mucin1-binding polypeptide. The mucin1-binding polypeptide is suitable for use in chimeric antigen receptors because it may be engineered to be expressed as part of a single chain along with other CAR components.

The chimeric antigen receptor may typically include an extracellular domain comprising the mucin1-binding polypeptide; a transmembrane domain; an intracellular signaling domain or a T cell activation domain. Further, the extracellular domain may further include a spacer region (or hinge region) between the polypeptide and the transmembrane domain. Further, the chimeric antigen receptor may further include one or more co-stimulatory domains, and preferably, the co-stimulatory domains may be placed between the transmembrane domain and the intracellular signaling domain.

Accordingly, in one preferred embodiment, the chimeric antigen receptor may include an extracellular domain comprising the mucin1-binding polypeptide; a hinge domain; a transmembrane domain; one or more co-stimulatory domains; and an intracellular signaling domain. Each domain may be heterogeneous. In other words, it may consist of sequences derived from different protein chains. Each domain may be linked by a short oligo- or polypeptide linker, for example, a linker having a length of 2 to 10 amino acids. In addition, the chimeric antigen receptor may consist of one polypeptide chain in which the respective domains are linked.

The extracellular domain includes the mucin1-binding polypeptide as described above, which recognizes mucin1 expressed on the cancer cell surface.

The extracellular domain may further include a spacer region (or hinge region). The spacer region may be placed between the mucin1-binding polypeptide and the transmembrane domain. The spacer region allows the mucin1-binding polypeptide to more flexibly recognize the target antigen at a predetermined distance from the cell membrane of CAR-T cell. The spacer region may be typically a polypeptide having a length of 10 or more amino acids, for example, a length of 10 to 300 amino acids, a length of 10 to 250 amino acids, a length of 10 to 200 amino acids, a length of 10 to 150 amino acids, a length of 10 to 100 amino acids, a length of 10 to 50 amino acids, but is not limited thereto.

The spacer region may be exemplified by a hinge region of CD8α or CD28, a constant region of an immunoglobulin (IgG), etc., and mutations may be introduced to remove off-target effects thereby. For example, the immunoglobulin constant region may be derived from an IgG hinge alone, a part or all of CH2 and CH3 domains, for example, an Fc region. IgG may be preferably IgG2 or IgG4. In a specific embodiment, the spacer may be a chimeric polypeptide containing one or more of hinge, CH2 and CH3 sequences derived from IgG2, IgG4, and/or IgG2 and IgG4. In a specific embodiment, the hinge domain may be represented by a sequence of SEQ ID NO: 112, but is not limited thereto.

The transmembrane domain serves to connect a cell membrane domain and a signaling domain inside the cell membrane, and may be derived from natural or synthetic sources. When derived from a natural source, the domain may be derived from any membrane-binding or transmembrane protein. For example, the transmembrane domain may be a transmembrane domain of a T cell receptor alpha, beta, or zeta chain, CD3 epsilon, CD4, CD5, CD8, CD9, CD16, CD22, CD28, CD33, CD37, CD45, CD64, CD80, CD86, CD134, CD137, or CD154, but is not limited thereto. In one preferred embodiment, the transmembrane domain may be a transmembrane domain of CD28 or CD8, but is not limited thereto. When derived from a synthetic source, the synthetic transmembrane domain may include hydrophobic residues, such as leucine and valine, and may include phenylalanine, tryptophan, and valine at each end thereof, but is not limited thereto. In one embodiment, the transmembrane domain may be represented by a sequence of SEQ ID NO: 113, but is not limited thereto.

The co-stimulatory domain is a site to which co-stimulatory signals are transmitted, and it is a site that transmits signals for CAR-T cells to trigger an immune response and proliferation. The co-stimulatory domain may be selectively introduced to improve CAR-T cell proliferation, cytotoxicity, sustained response, and lifespan extension. The co-stimulatory domain may be one or more, for example, one, two, or three selected from signaling domains of CD28, OX-40 (CD134), 4-1BB (CD137), CD2, CD7, CD27, CD30, CD40, PD-1, ICOS, LFA-1 (CD11a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class I molecule, TNF receptor protein, immunoglobulin protein, cytokine receptor, integrin, signaling lymphocytic activation molecule (SLAM), activating NK cell receptor, BTLA, Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8 beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAMF1 (CD150, IPO-3), BLAME (SLAMF8), SELPLG (CD162), LTBR, LAT, GADS, SLP-76, PAG/Cbp, or CD19a, but is not limited thereto. In one preferred embodiment, the co-stimulatory domain may be one or more, for example, one, two, or three selected from signaling domains of CD28, OX-40 (CD134), 4-1BB (CD137), CD27, or ICOS, but is not limited thereto. In an embodiment, the co-stimulatory domain may be represented by a sequence of SEQ ID NO: 114, but is not limited thereto.

The intracellular signaling domain is a site that activates a T cell immune response to an antigen bound to the mucin1-binding polypeptide. The intracellular signaling domain may be a component of the T cell receptor (TCR) or may include a signaling motif known as an immunoreceptor tyrosine-based activation motif (ITAM). For example, the intracellular signaling domain may be a signaling domain derived from TCR or CD3 zeta, FcR gamma, CD3 gamma, CD3 delta and CD3 epsilon, but is not limited thereto. For a preferred example, the intracellular signaling domain may be a signaling domain of CD3 zeta, but is not limited thereto. The intracellular signaling domain may activate CAR-T cells when the mucin1-binding polypeptide region of the extracellular domain binds to a target. For example, CAR may stimulate T cell function, e.g., cell lytic activity or T-helper activity, and may induce secretion of cytokines or other factors. In some embodiments, the intracellular signaling domain may be represented by a sequence of SEQ ID NO: 115, but is not limited thereto.

In some embodiments, CAR may be expressed in a form comprising a signal sequence. In addition, CAR may be expressed together with additional sequences useful for monitoring expression, for example, a ribosomal skip sequence such as 2A peptide or a truncated cell surface polypeptide (tHER2 or tEGFR or truncated PSMA, etc.).

According to still another aspect of the present invention, provided is an isolated polynucleotide encoding the chimeric antigen receptor. In some embodiments, the polynucleotide may be codon optimized for expression in humans.

According to still another aspect of the present invention, provided is a vector comprising the polynucleotide encoding the chimeric antigen receptor. The vector may be constructed as a vector for gene cloning, a vector for protein expression, or a vector for gene delivery. The details of the vector for cloning or expression are as described above.

Any vector known in the art may be suitable for the present invention. For example, the vector may be a viral vector. For example, the vector is a retrovirus vector, a DNA vector, a murine leukemia virus vector, an SFG vector, a plasmid, an RNA vector, an adenovirus vector, a baculovirus vector, an Epstein Barr virus vector, a papovavirus vector, a vaccinia virus vector, a herpes simplex virus vector, an adenovirus-associated vector (AAV), or a lentivirus vector, but is not limited thereto.

According to still another aspect of the present invention, provided is an immune cell expressing the chimeric antigen receptor comprising the mucin1-binding polypeptide.

The immune cell may include T cells, tumor infiltrating lymphocytes (TIL), natural killer (NK) cells, TCR-expressing cells, dendritic cells, or NK-T cells, but is not limited thereto. In addition, the immune cell may be derived from human induced pluripotent stem cells (iPSCs). The immune cell may be derived from any known source. For example, the immune cell may be differentiated in vitro from a population of hematopoietic stem cells, or obtained from a patient. The immune cell may be obtained from, for example, peripheral blood mononuclear cells (PBMC), bone marrow, a lymph node tissue, umbilical cord blood, a thymus tissue, a tissue from an infection site, ascites, pleural effusion, a splenic tissue, and a tumor. In addition, the immune cell may be derived from one or more immune cell lines available in the related art.

The immune cell may be autologous or allogenic. Autologous means those derived from a patient to be treated. Allogeneic means those derived from another individual of the same species as the patient to be treated.

In addition, the immune cell may be derived from human induced pluripotent stem cells (iPSCs). As compared with autologous or allogeneic immune cells, iPSC-derived immune cells may self-proliferate. Thus, mass-proliferation thereof is easy, and it is possible to prepare a general-purpose cell therapy applicable to all people, and there are advantages of homogeneous production and cost reduction.

The immune cell may be transfected or transduced by a vector using a method of microinjection, electroporation, sonication, biolistics (e.g., gene gun), lipid transfection, polymer transfection, calcium phosphate precipitation, protoplast fusion, liposome-mediated transfection, nanoparticles or polyplexes, etc., but is not limited thereto.

According to still another aspect, provided is a composition for preventing or treating cancer, the composition comprising the mucin1-binding polypeptide; the isolated polynucleotide encoding the mucin1-binding polypeptide; the vector comprising the polynucleotide encoding the mucin1-binding polypeptide; the cell comprising the polynucleotide encoding the mucin1-binding polypeptide; the chimeric antigen receptor comprising the mucin1-binding polypeptide; the isolated polynucleotide encoding the chimeric antigen receptor; the vector comprising the polynucleotide encoding the chimeric antigen receptor; or the immune cell comprising the polynucleotide encoding the chimeric antigen receptor, or expressing the chimeric antigen receptor.

According to still another aspect, provided is a method of preventing or treating cancer, the method comprising administering, to a patient in need of preventing or treating cancer, an effective amount of the mucin1-binding polypeptide; the isolated polynucleotide encoding the mucin1-binding polypeptide; the vector comprising the polynucleotide encoding the mucin1-binding polypeptide; the cell comprising the polynucleotide encoding the mucin1-binding polypeptide; the chimeric antigen receptor comprising the mucin1-binding polypeptide; the isolated polynucleotide encoding the chimeric antigen receptor; the vector comprising the polynucleotide encoding the chimeric antigen receptor; or the immune cell comprising the polynucleotide encoding the chimeric antigen receptor, or expressing the chimeric antigen receptor.

According to still another aspect, provided is a composition for preventing or treating cancer, the composition comprising T cells comprising the chimeric antigen receptor comprising the mucin1-binding polypeptide.

According to still another aspect, provided is a method of preventing or treating cancer, the method comprising administering, to a patient in need of preventing or treating cancer, an effective amount of the T cells comprising the chimeric antigen receptor comprising the mucin1-binding polypeptide.

For example, the cancer may be a solid cancer or a blood cancer. Non-limiting examples thereof may include breast cancer, lung cancer, prostate cancer, ovarian cancer, brain cancer, liver cancer, cervical cancer, endometrial cancer, uterine cancer, colon cancer, colorectal cancer, colorectal cancer, rectal cancer, kidney cancer, nephroblastoma, skin cancer, oral squamous cell carcinoma, epidermoid carcinoma, nasopharyngeal cancer, head and neck cancer, bone cancer, esophageal cancer, bladder cancer, lymphoma (e.g., Hodgkin's lymphoma or non-Hodgkin's lymphoma), gastric cancer, pancreatic cancer, testicular cancer, thyroid cancer, follicular carcinoma, melanoma, myeloma, multiple myeloma, mesothelioma, osteosarcoma, myelodysplastic syndrome, tumor of mesenchymal origin, soft tissue sarcoma, liposarcoma, gastrointestinal stromal sarcoma, malignant peripheral nerve sheath tumor (MPNST), Ewing's sarcoma, leiomyosarcoma, mesenchymal chondrosarcoma, lymphosarcoma, fibrosarcoma, rhabdomyosarcoma, teratocarcinoma, neuroblastoma, medulloblastoma, glioma, benign skin tumor, or leukemia. The lung cancer may be, for example, small cell lung carcinoma (SCLC) or non-small cell lung carcinoma (NSCLC). The leukemia may be, for example, acute myeloid leukemia (AML), chronic myelogenous leukemia (CML), acute lymphocytic leukemia (ALL), or chronic lymphocytic leukemia (CLL).

Preferably, the cancer may be MUC1 protein-expressing cancer, and it may be breast cancer, skin cancer, pancreatic cancer, prostate cancer, lung cancer, thyroid cancer, gastric cancer, ovarian cancer, colorectal cancer, liver cancer, gallbladder cancer, kidney cancer, cervical cancer, or bladder cancer, but is not limited thereto. The cancer may be primary or metastatic cancer.

The patient to be treated may be a patient receiving a secondary anti-hyperproliferative therapy. For example, the secondary anti-hyperproliferative therapy may be chemotherapy, radiation therapy, immunotherapy, phototherapy, cryotherapy, toxin therapy, hormone therapy, or surgery.

CAR-T anticancer therapy using T cells including the chimeric antigen receptor including the mucin1-binding polypeptide of the present disclosure may be performed by a series of processes including extracting T cells from the blood of a healthy person or a patient to be treated, genetically engineering the T cells to express the chimeric antigen receptor including the mucin1-binding polypeptide of the present disclosure, expanding and culturing the engineered T cells, and administering the engineered T cells thus cultured to the patient.

In a preferred embodiment, the step of extracting T cells from the blood of a healthy person or a patient to be treated may be performed by extracting T cells after isolating leukocytes from the blood using leukapheresis or aphresis and enriching T cells. T cells may be separated using specific antibody bead conjugates or markers at the level of CD4/CD8 composition. Alternatively, it is also possible to stably obtain a large amount of T cells through differentiation from stem cells.

The step of genetically engineering the extracted T cells to express the chimeric antigen receptor provided in the present disclosure may be performed by, for example, injecting a nucleotide molecule designed to express the chimeric antigen receptor (CAR) into T cells using a vector, for example, a viral vector (a lentivirus vector, a retrovirus vector, etc.). CAR may be introduced in the form of DNA, or CAR may be introduced in the form of RNA, RNA is then reverse-transcribed into DNA by reverse transcriptase, which may be integrated into the genome of T cells. In addition, in the case of allogeneic cell therapy, genetic manipulation to remove or correct genes inducing graft rejection in donor T cells may be additionally performed to prevent rejection, such as graft-versus-host disease (GvHD).

The step of expanding and culturing the engineered T cells may be performed by culturing, proliferating, and expanding T cells according to a culture technique known in the art. In this regard, safety against virus use and a technology for selecting well-made CAR-T cells are required.

Lastly, the step of administering the engineered T cells to the patient may be performed by, for example, infusion. In a preferred embodiment, before CAR-T cell injection, the patient may receive a lymphodepletion chemotherapy with cyclophosphamide or fludarabine to reduce the level of white blood cells. In addition, to improve the persistence of CAR-T cells, cytokines such as IL-2 may be administered together.

The engineered T cells administered to the patient may mediate immune responses against mucin1-expressing tumor cells. These immune responses include activation of T cells, secretion of cytokines such as IL-2 and IFN-gamma by T cells, proliferation and expansion of T cells recognizing tumor antigens, and T cell-mediated specific killing (removal of tumor) of target-positive cells. For example, when CAR specifically binds to mucin1 in CAR-T cells, T cells are activated through phosphorylation of an immunoreceptor tyrosine-based activation motif (ITAM) of CD3 zeta, and subsequently, T cell proliferation, cytotoxicity and/or cytokine secretion may be induced.

As described above, the anticancer therapy using CAR-T cells fundamentally activates the immune system of a patient and exerts a continuous anticancer effect, and thus, it is advantageous in that there is no need to continue administration, and personalized treatment is possible by using the patient's own T cells.

Administration of the composition of the present disclosure may cause, induce, or promote an immune response that inhibits, stops, delays, or prevents the onset or progression of a disease.

The term “effective amount” refers to an amount sufficient to achieve the desired result, e.g., an amount effective to treat or prevent cancer, when administered to subjects, including humans. The effective amount may vary depending on various factors such as a formulation method, administration mode, a patient's age, body weight, sex, disease severity, diet, administration time, administration route, excretion rate, and response sensitivity. Administration dosage or therapeutic regimen may be adjusted to provide an optimal therapeutic response as will be understood by those skilled in the art.

A therapeutic regimen with a therapeutically effective amount may consist of a single-dose administration, or alternatively, may include a series of applications. The duration of the treatment phase is determined by various factors such as a formulation method, administration mode, a patient's age, body weight, sex, disease severity, diet, administration time, administration route, excretion rate, and response sensitivity. It will also be appreciated that the effective dosage of the anti-cancer composition used for treatment may be increased or lowered during the course of an individual therapeutic regimen. Variations in dosage may occur and will be apparent through standard diagnostic analysis known in the art. The composition of the present invention, in some aspects, may be administered before, during, or after treatment with common anticancer agents, radiation therapy, hormone therapy, biotherapy and/or surgical resection of tumor.

The composition of the present disclosure may be provided together with one or more additives selected from the group consisting of pharmaceutically acceptable carriers, diluents, and excipients.

The pharmaceutically acceptable carrier, which is commonly used in the formulation of antibody, may be one or more selected from the group consisting of lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, mineral oil, etc., but is not limited thereto. The composition may further include one or more selected from the group consisting of diluents, excipients, lubricants, wetting agents, sweeteners, flavoring agents, emulsifiers, suspending agents, preservatives, etc., which are commonly used in the preparation of pharmaceutical compositions, in addition to the above components.

The composition may be administered orally or parenterally. When administered parenterally, intravenous injection, subcutaneous injection, intramuscular injection, intraperitoneal injection, endothelial administration, topical administration, intranasal administration, intrapulmonary administration and rectal administration may be used. When administered orally, proteins or peptides are digested, and thus compositions for oral administration may be formulated to coat the active agent or to protect it from degradation in the stomach. In addition, the composition may be administered by any device capable of transporting the active substance to a target cell.

The cells may be administered using standard administration techniques, formulations, or devices. Formulations and devices such as syringes and vials for storage and administration of the composition may be appropriately used. Administration of cells may be autologous, allogeneic, or xenogenic. Peripheral blood-derived immune cells or progeny thereof (e.g., derived in vivo, ex vivo, or in vitro) may be administered via catheter administration, systemic injection, local injection, intravenous injection, or local injection including parenteral administration. When immune cell therapeutics are administered, they may be generally formulated into injectable unit dosage forms (solutions, suspensions, emulsions). In some embodiments, a population of cells may be administered parenterally. Formulations include those for intravenous injection, subcutaneous injection, intramuscular injection, intraperitoneal injection, endothelial administration, topical administration, intranasal administration, intrapulmonary administration, and rectal administration.

In some embodiments, the composition is provided as a sterile liquid preparation, for example, as an isotonic aqueous solution, a suspension, an emulsion, a dispersion, or a viscous composition, which, in some aspects, may be buffered to a selected pH. Liquid preparations are normally easier to prepare than gels, other viscous compositions, and solid compositions. In addition, liquid compositions are particularly convenient to administer by injection. Viscous compositions, on the other hand, may be formulated within the appropriate viscosity range to provide longer contact periods with a specific tissue. Liquid or viscous compositions may include a carrier which may be a solvent or dispersion medium containing, for example, water, saline, phosphate buffered saline, polyol (e.g., glycerol, propylene glycol, liquid polyethylene glycol) and appropriate mixtures thereof.

Sterile injectable solutions may be prepared by incorporating the binding molecule in a solvent, such as in admixture with a suitable carrier, diluent, or excipient such as sterile water, physiological saline, glucose, dextrose, or the like. The compositions may also be lyophilized. The compositions may include auxiliary substances such as wetting, dispersing, or emulsifying agents (e.g., methylcellulose), pH buffering agents, gelling or viscosity enhancing additives, preservatives, flavoring agents, colors, etc., depending upon the route of administration and the desired preparation.

Various additives which enhance stability and sterility of the compositions, including antimicrobial preservatives, antioxidants, chelating agents, and buffers, may be added. Prevention of microbial actions may be ensured by various antibacterial and antifungal agents, for example, parabens, chlorobutanol, phenol, sorbic acid, etc. Prolonged absorption of the injectable pharmaceutical form may be brought about by the use of agents delaying absorption, for example, aluminum monostearate and gelatin.

Hereinafter, the present invention will be described in more detail with reference to the following exemplary embodiments. However, these exemplary embodiments are only for illustrating the present invention, and the scope of the present invention is not limited by these exemplary embodiments.

### Example 1. Preparation of scFv Binding to Mucin1

Human mucin1 (SEQ ID NO: 110) was conjugated with CD4 (SEQ ID NO: 111) to prepare a recombinant antigen. From scFv libraries, clones secreting scFv specific to the antigen were screened, and 10 kinds of scFvs specifically binding to mucin1 were selected. Sequence information of 10 kinds of scFvs thus selected and a positive control scFv G03 (a positive control prepared using an amino acid sequence of G3 antibody of Table 6 of WO2018/174544) used in subsequent experiments are shown in Table 3.

TABLE 3

| Clone | |
|---|---|
| name | scFv amino acid sequence |
| 2445_ | EVQLLETGGGLIQPGGSLRLSCAASGFTVSSNYMSWVRQAPGKGLEWVS |
| 1F09 | VIYSGGSTYYADSVKGRFTISRDNSKNTLYLQMNSLRAEDTAVYYCARDR |
| | GWNHGMDVWGQGTLVTVSSGGGGSGGGGSGGGASQSGLTQPPSASGTP |
| | GQRVTISCSGGSSEVGSRYVSWYQQLPGTAPRLLIYKNDRRPSGVPDRESG |
| | SKSGSSASLAISGLRSEDEADYYCAAWDDSLNGYVFGTGTKLTVL (SEQ |
| | ID NO: 9) |
| 2447_ | EVQLVQSGAEVKKPGASVKVSCKASGYTFTNYGISWVRQAPGQGLEWM |
| 2H08 | GWISAYNGNTNYAQKLQGRVTMTTDTSTSTAYMELRSLRSDDTAVYYCA |
| | RDPHILTGYYRGGWFDPWGQGTTVTVSSGGGGSGGGGSGGGASSYELTQ |
| | DPAVSVALGQTVTITCQGDSLRTSYAGWLQQKPGQAPVLVLYGKTSRPSG |
| | IPDRESGSTSGNTASLTITGAQAEDEAEYFCHSRDSNDNYLEVVFGGGTKL |
| | TVL (SEQ ID NO: 19) |
| 2447_ | EVQLLESGGGLVQPGGSLRLSCAASGFTFDDYAMHWVRQAPGKGLEWV |
| 3A01 | SGISWNSGSIVYADFVKGRFTISRDNAKNSLYLQMNSLRAEDTALYYCAK |
| | DISSGWYPGTEDYWGQGTLVTVSSGGGGSGGGGSGGGASSYELTQDPAV |
| | SVALGQTVRITCQGDSLRSYYASWYQQKPGQAPVLVIYGKNNRPSGIPDR |
| | FSGSSSGNTASLTVTGAQAEDEADYYCSSRDSSDDVVFGGGTQLTVL |
| | (SEQ ID NO: 29) |
| 2447_ | EVQLVQSGAEVKKPGASVKVSCKASGYTFTSYGISWVRQAPGQGLEWM |
| 3A08 | GWISAYNGNTNYAQKLQGRVTMTTDTSTSTAYMELRSLRSDDTAVYYCA |
| | RDRATIFGVVTPFDYWGQGTLVTVSSGGGGSGGGGSGGGASSYELTQDP |
| | AVSVALGQTVRITCQGDSIRSYSASWYQQKPGQAPRLVIYGKNNRPSGIPD |
| | RVSGSTSGNTASLTVTGAQAEDEADYYCNSRDSSGNRVVFGGGTQLTVL |
| | (SEQ ID NO: 39) |
| 2447_ | EVQLLESGGGLVQPGRSLRLSCAATGFTFDDYAMHWVRQAPGKGLEWV |
| 3A09 | SGISWNSGSIGYADSVKGRFTISRDNAKNSLYLQMNSLRAEDTASYYCAK |
| | DVSSGWYWYAFDIWGQGTLVTVSSGGGGSGGGGSGGGASSYELTQDPA |
| | VSVALGQTVRITCQGDSLRSYYASWYQQKPGQAPVLVIYGKNNRPSGIPG |
| | RFSGSSSGNTASLTVTGAQAEDEADYYCNSRDSGGSVVFGGGTKLTVL |
| | (SEQ ID NO: 49) |
| 2447_ | EVQLVQSGAEVKKPGASVKVSCKASGYTFTSYGISWVRQAPGQGLEWM |
| 3A12 | GWISAYNGNTNYAQKLRGRVTMTTDTSTSTAYMELRSLRSDDTAVYYCA |
| | RDPHILTGYYRGGWFDPWGQGTLVTVSSGGGGSGGGGSGGGASSYELTQ |
| | DPAVSVVLGQTVRITCQGDSLRSYYASWYQQKPGQAPVLVIYGKNNRPS |
| | GIPDRFSGSSSGNTASLTITGAQAEDEADYYCNSRDSSGNHRVFGGGTKLT |
| | VL (SEQ ID NO: 59) |
| 2447_ | QVQLVESGGVVVQPGGSLRLSCAASGFTFDDYAMHWVRQAPGRGLEWV |
| 3B07 | SLTSWDGGSTYYADSVKGRFTISRDNSKNSLYLQMNSLRAEDTALYYCA |
| | KDHSSGWYNGGMDVWGQGTMVTVSSGGGGSGGGGSGGGASSYELTQD |
| | PAVSVALGQTVRITCQGDSLRSYYASWYQQKPGQAPVLVIYGKNNRPSGI |
| | PDRFSGSSSGNTASLTITGAQAEDEADYYCNSRDSSGNHVVFGGGTKLTV |
| | L (SEQ ID NO: 69) |
| 2447_ | EVQLLESGGGLVQPGGSLRLSCAASGFTFDDYAMHWVRQAPGKGLEWV |
| 3C08 | SGISWNSGSIGYADSVKGRFTISRDNAKNSLYLQMNSLRAEDTALYYCAK |
| | DRGSGYEGNYYGMDVWGQGTLVTVSSGGGGSGGGGSGGGASSYELTQD |
| | PAVSVALGQTVRITCQGDSLRSYYASWYQQKPGQAPVLVIYGKNNRPSGI |
| | PDRFSGTTSGNTASLTITGAQAEDEADYYCNSRDSSGNHYVFGTGTKVTV |
| | L (SEQ ID NO: 79) |
| 2447_ | EVQLLESGGGLVQPGRSLRLSCAASGFTFDDYAMHWVRQAPGKGLEWVS |
| 3H02 | GISWDSGSIGYADSVKGRFTISRDNAKNSLYLQMNSLRAEDTALYYCAKD |
| | VSSGWYWYAFDIWGQGTLVTVSSGGGGSGGGGSGGGASSYELTQDPAVS |
| | VALGQTVRITCQGDILRSYYASWYQQKPGQAPVLVIYGKNNRPSGIPDRFS |
| | GSSSGNTASLTITGAQAEDEADYYCNSRDSSGNRVVFGGGTKLTVL (SEQ |
| | ID NO: 89) |
| 2447_ | EVQLLESGGGLVQPGRSLRLSCAASGFTFDDYAMHWVRQAPGKGLEWVS |
| 3H08 | GISWNSGSIDYADSVKGRFTISRDNSKNTLYLQMNSLRAEDTAVYYCARD |
| | RSSGWYTGSFDYWGQGTLVTVSSGGGGSGGGGSGGGASSYELTQDPAVS |
| | VALGQTVRITCQGDSLRSYYASWYRQKPGQAPVLVIYGKNNRPSGIPDRF |
| | SGSSSGNTASLTIIGAQAEDEADYYCQSRDSSDNRVLFGGGTKVTVL (SEQ |
| | ID NO: 99) |
| G03 | EVQLVQSGAEVKKPGATVKISCKVSGYTFTSYWMHWVQQAPGKGLEWI |
| | GYINPGTGYIEYNQKFKDRVTITADKSTDTAYMELSSLRSEDTAVYYCASS |
| | TAPFDYWGQGTLVTVSSGGGGSGGGGSGGGASEIVLTQSPGTLSLSPGER |
| | ATLSCKASQDIKSYLSWYQQKPGQAPRLLIYYATRLADGIPDRESGSGSGT |
| | DFTLTISRLEPEDFAVYYCLQYDESPYTFGQGTKLEIK (SEQ ID NO: 109) |

TABLE 4

| Clone | |
|---|---|
| name | scFv nucleotide sequence |
| 2445_ | GAGGTGCAGCTGTTGGAGACTGGAGGAGGCTTGATCCAGCCTGGGGG |
| 1F09 | GTCCCTGAGACTCTCCTGTGCAGCCTCTGGGTTCACCGTCAGTAGCAAC |
| | TACATGAGCTGGGTCCGCCAGGCTCCAGGGAAGGGGCTGGAGTGGGTC |
| | TCAGTTATTTATAGCGGTGGTAGCACATATTACGCAGACTCCGTGAAG |
| | GGCCGATTCACCATCTCCAGAGACAATTCCAAGAACACGCTGTATCTT |
| | CAAATGAACAGCCTGAGAGCCGAGGACACGGCCGTGTATTACTGTGCG |
| | AGAGATCGCGGCTGGAACCACGGTATGGACGTCTGGGGCCAGGGAAC |
| | CCTGGTCACCGTCTCGAGTGGTGGAGGCGGTTCAGGCGGAGGTGGCTC |
| | TGGCGGTGGCGCTAGCCAGTCTGGGCTGACTCAGCCACCCTCAGCGTC |
| | TGGGACCCCCGGGCAGAGGGTCACCATCTCTTGTTCTGGGGGCAGCTC |
| | AGAAGTCGGAAGTAGATATGTGTCCTGGTACCAGCAACTCCCAGGAAC |
| | GGCCCCCAGACTCCTCATCTATAAGAATGATCGGCGGCCCTCAGGGGT |
| | CCCTGACCGATTCTCTGGCTCCAAGTCTGGCTCCTCAGCCTCCCTGGCC |
| | ATCAGTGGGCTCCGGTCCGAGGATGAGGCTGATTATTACTGTGCAGCA |
| | TGGGATGACAGCCTGAATGGTTATGTCTTCGGAACTGGGACCAAGCTG |
| | ACCGTCCTA (SEQ ID NO: 10) |
| 2447_ | GAAGTGCAGCTGGTGCAGTCTGGAGCTGAGGTGAAGAAGCCTGGGGC |
| 2H08 | CTCAGTGAAGGTCTCCTGCAAGGCTTCTGGTTACACCTTTACCAACTAC |
| | GGTATCAGCTGGGTGCGACAGGCCCCTGGACAAGGGCTTGAGTGGATG |
| | GGATGGATCAGCGCTTACAATGGTAACACAAACTATGCACAGAAGCTC |
| | CAGGGCAGAGTCACCATGACCACAGACACATCCACGAGCACAGCCTA |
| | CATGGAGCTGAGGAGCCTGAGATCTGACGACACGGCCGTGTATTACTG |
| | TGCGAGAGATCCGCATATTTTGACTGGTTATTATAGGGGAGGGTGGTT |
| | CGACCCCTGGGGGCAAGGGACCACGGTCACCGTCTCGAGTGGTGGAG |
| | GCGGTTCAGGCGGAGGTGGCTCTGGCGGTGGCGCTAGCTCCTATGAGC |
| | TGACTCAGGACCCTGCTGTGTCTGTGGCCTTGGGTCAGACAGTCACCAT |
| | CACGTGCCAAGGAGACAGCCTCAGAACCTCTTATGCAGGCTGGCTCCA |
| | GCAGAAGCCAGGACAGGCCCCTGTACTCGTCCTCTATGGTAAAACCAG |
| | CCGGCCCTCAGGGATCCCAGACCGATTCTCTGGCTCCACCTCAGGAAA |
| | CACAGCTTCCTTGACCATCACTGGGGCTCAGGCGGAGGATGAGGCTGA |
| | GTATTTCTGTCACTCCCGGGACAGCAATGATAACTATCTAGAGGTGGTT |
| | TTCGGCGGAGGGACCAAGCTGACCGTCCTA (SEQ ID NO: 20) |
| 2447_ | GAGGTGCAGCTGTTGGAGTCTGGGGGAGGCTTGGTACAGCCTGGGGGG |
| 3A01 | TCCCTGAGACTCTCCTGTGCAGCCTCTGGATTCACCTTTGATGATTATG |
| | CCATGCACTGGGTCCGGCAAGCTCCAGGGAAGGGCCTGGAGTGGGTCT |
| | CAGGTATTAGTTGGAATAGTGGTAGCATAGTCTATGCGGACTTTGTGA |
| | AGGGCCGATTCACCATCTCCAGAGACAACGCCAAGAACTCCCTGTATC |
| | TGCAAATGAACAGTCTGAGAGCTGAGGACACGGCCTTGTATTACTGTG |
| | CAAAAGATATTTCAAGTGGCTGGTACCCAGGGACCTTTGACTACTGGG |
| | GCCAGGGAACCCTGGTCACCGTCTCGAGTGGTGGAGGCGGTTCAGGCG |
| | GAGGTGGCTCTGGCGGTGGCGCTAGCTCCTATGAGCTGACTCAGGACC |
| | CTGCTGTGTCTGTGGCCTTGGGACAGACAGTCAGGATCACATGCCAAG |
| | GAGACAGCCTCAGAAGCTATTATGCAAGCTGGTACCAGCAGAAGCCA |
| | GGACAGGCCCCTGTACTTGTCATCTATGGTAAAAACAACCGGCCCTCA |
| | GGGATCCCAGACCGATTCTCTGGCTCCAGCTCAGGAAACACAGCTTCC |
| | TTGACCGTCACTGGGGCTCAGGCGGAAGACGAGGCTGACTATTACTGT |
| | AGCTCCCGGGACAGCAGTGATGATGTGGTATTCGGCGGAGGGACCCAG |
| | CTCACCGTCCTA (SEQ ID NO: 30) |
| 2447_ | GAAGTGCAGCTGGTGCAGTCTGGAGCTGAGGTGAAGAAGCCTGGGGC |
| 3A08 | CTCAGTGAAGGTCTCCTGCAAGGCTTCTGGTTACACCTTTACCAGCTAC |
| | GGTATCAGCTGGGTGCGACAGGCCCCTGGACAAGGGCTTGAGTGGATG |
| | GGATGGATCAGCGCTTACAATGGTAACACAAACTATGCACAGAAGCTC |
| | CAGGGCAGAGTCACCATGACCACAGACACATCCACGAGCACAGCCTA |
| | CATGGAGCTGAGGAGCCTGAGATCTGACGACACGGCCGTGTATTACTG |
| | TGCGAGAGATCGGGCTACGATTTTTGGAGTGGTTACCCCCTTTGACTAC |
| | TGGGGCCAAGGAACCCTGGTCACCGTCTCGAGTGGTGGAGGCGGTTCA |
| | GGCGGAGGTGGCTCTGGCGGTGGCGCTAGCTCCTATGAGCTGACTCAG |
| | GACCCCGCTGTGTCTGTGGCCTTGGGACAGACAGTCAGGATCACATGC |
| | CAGGGAGACAGTATTAGAAGTTATTCTGCCAGCTGGTACCAGCAGAAG |
| | CCAGGGCAGGCCCCTCGCCTTGTTATCTATGGTAAAAACAACCGGCCC |
| | TCAGGGATCCCAGACCGAGTCTCTGGCTCCACTTCAGGAAATACAGCT |
| | TCCTTGACCGTCACTGGGGCTCAGGCGGAAGATGAGGCTGACTATTAC |
| | TGTAACTCCCGGGACAGCAGTGGTAACCGTGTGGTATTCGGCGGAGGG |
| | ACCCAGCTCACCGTCCTA (SEQ ID NO: 40) |
| 2447_ | GAGGTGCAGCTGTTGGAGTCTGGGGGAGGCTTGGTACAGCCTGGCAGG |
| 3A09 | TCCCTGAGACTCTCCTGTGCAGCCACTGGATTCACCTTTGATGATTATG |
| | CCATGCACTGGGTCCGGCAAGCTCCAGGGAAGGGCCTGGAGTGGGTCT |
| | CAGGTATTAGTTGGAATAGTGGTAGCATAGGCTATGCGGACTCTGTGA |
| | AGGGCCGATTCACCATCTCCAGAGACAACGCCAAGAACTCCCTGTATC |
| | TGCAAATGAACAGTCTGAGAGCTGAGGACACGGCCTCGTATTACTGTG |
| | CAAAAGATGTTAGCAGTGGCTGGTACTGGTATGCTTTTGATATCTGGG |
| | GCCAAGGAACCCTGGTCACCGTCTCGAGTGGTGGAGGCGGTTCAGGCG |
| | GAGGTGGCTCTGGCGGTGGCGCTAGCTCCTATGAGCTGACTCAGGACC |
| | CTGCTGTGTCTGTGGCCTTGGGACAGACAGTCAGGATCACATGCCAAG |
| | GAGACAGCCTCAGAAGCTATTATGCAAGCTGGTACCAGCAGAAGCCA |
| | GGACAGGCCCCTGTACTTGTCATCTATGGTAAAAACAACCGGCCCTCA |
| | GGGATCCCAGGCCGATTCTCTGGCTCCAGCTCAGGAAACACAGCTTCC |
| | TTGACCGTCACTGGGGCTCAGGCGGAAGATGAGGCTGACTATTACTGT |
| | AATTCCCGGGACAGCGGTGGTTCTGTGGTTTTCGGCGGAGGGACCAAG |
| | CTGACCGTCCTA (SEQ ID NO: 50) |
| 2447_ | GAAGTGCAGCTGGTGCAGTCTGGAGCTGAGGTGAAGAAGCCTGGGGC |
| 3A12 | CTCAGTGAAGGTCTCCTGCAAGGCTTCTGGTTACACCTTTACCAGCTAT |
| | GGTATCAGCTGGGTGCGACAGGCCCCTGGACAAGGGCTTGAGTGGATG |
| | GGATGGATCAGCGCTTACAATGGTAACACAAACTATGCACAGAAGCTC |
| | CGGGGCAGAGTCACCATGACCACAGACACATCCACGAGCACAGCCTA |
| | CATGGAGCTGAGGAGCCTGAGATCTGACGACACGGCCGTGTATTACTG |
| | TGCGAGAGATCCGCATATTTTGACTGGTTATTATAGGGGAGGGTGGTT |
| | CGACCCCTGGGGCCAGGGCACCCTGGTCACCGTCTCGAGTGGTGGAGG |
| | CGGTTCAGGCGGAGGTGGCTCTGGCGGTGGCGCTAGCTCCTATGAGCT |
| | GACTCAGGACCCTGCTGTGTCTGTGGTCTTGGGACAGACAGTCAGGAT |
| | CACATGCCAAGGAGACAGCCTCAGAAGCTATTATGCAAGCTGGTACCA |
| | GCAGAAGCCAGGACAGGCCCCTGTACTTGTCATCTATGGTAAAAACAA |
| | CCGGCCCTCAGGGATCCCAGACCGATTCTCTGGCTCCAGCTCAGGAAA |
| | CACAGCTTCCTTGACCATCACTGGGGCTCAGGCGGAAGATGAGGCTGA |
| | CTATTACTGTAACTCCCGGGACAGCAGTGGTAACCATCGTGTATTCGG |
| | CGGAGGGACCAAGCTGACCGTCCTA (SEQ ID NO: 60) |
| 2447_ | CAGGTGCAGCTGGTGGAGTCTGGGGGAGTCGTGGTACAGCCTGGGGG |
| 3B07 | GTCCCTGAGACTCTCCTGTGCAGCCTCTGGATTCACCTTTGATGATTAT |
| | GCCATGCACTGGGTCCGTCAAGCTCCGGGGAGGGGTCTGGAGTGGGTC |
| | TCTCTTACTAGTTGGGATGGTGGTAGCACATACTATGCAGACTCTGTGA |
| | AGGGTCGATTCACCATCTCCAGAGACAACAGCAAAAACTCCCTGTATC |
| | TGCAAATGAACAGTCTGAGAGCTGAGGACACCGCCTTGTATTACTGTG |
| | CAAAAGATCATAGCAGCGGCTGGTACAACGGGGGTATGGACGTCTGG |
| | GGCCAAGGGACAATGGTCACCGTCTCGAGTGGTGGAGGCGGTTCAGGC |
| | GGAGGTGGCTCTGGCGGTGGCGCTAGCTCCTATGAGCTGACTCAGGAC |
| | CCTGCTGTGTCTGTGGCCTTGGGACAGACAGTCAGGATCACATGCCAA |
| | GGAGACAGCCTCAGAAGCTATTATGCAAGCTGGTACCAGCAGAAGCC |
| | AGGACAGGCCCCTGTACTTGTCATCTATGGTAAAAACAACCGGCCCTC |
| | AGGGATCCCAGACCGATTCTCTGGCTCCAGCTCAGGAAACACAGCTTC |
| | CTTGACCATCACTGGGGCTCAGGCGGAAGATGAGGCTGACTATTACTG |
| | TAACTCCCGGGACAGCAGTGGTAACCATGTGGTATTCGGCGGAGGGAC |
| | CAAGCTGACCGTCCTA (SEQ ID NO: 70) |
| 2447_ | GAGGTGCAGCTGTTGGAGTCTGGGGGAGGCTTGGTACAGCCTGGGGGG |
| 3C08 | TCCCTGAGACTCTCCTGTGCAGCCTCTGGATTCACCTTTGATGATTATG |
| | CCATGCACTGGGTCCGGCAAGCTCCAGGGAAGGGCCTGGAGTGGGTCT |
| | CAGGTATTAGTTGGAATAGTGGTAGCATAGGCTATGCGGACTCTGTGA |
| | AGGGCCGATTCACCATCTCCAGAGACAACGCCAAGAACTCCCTGTATC |
| | TGCAAATGAACAGTCTGAGAGCTGAGGACACGGCCTTGTATTACTGTG |
| | CAAAAGATAGGGGTAGTGGCTACGAAGGAAACTACTACGGTATGGAC |
| | GTCTGGGGCCAAGGAACCCTGGTCACCGTCTCGAGTGGTGGAGGCGGT |
| | TCAGGCGGAGGTGGCTCTGGCGGTGGCGCTAGCTCCTATGAGCTGACT |
| | CAGGACCCTGCTGTGTCTGTGGCCTTGGGACAGACAGTCAGGATCACA |
| | TGCCAAGGAGACAGCCTCAGAAGCTATTATGCAAGCTGGTACCAGCAG |
| | AAGCCAGGACAGGCCCCTGTACTTGTCATCTATGGTAAAAACAACCGG |
| | CCCTCAGGGATCCCAGACCGATTCTCTGGTACCACCTCAGGAAACACA |
| | GCCTCCTTGACCATCACTGGGGCTCAGGCGGAAGATGAGGCTGACTAT |
| | TACTGTAACTCCCGGGACAGCAGTGGTAACCATTATGTCTTCGGAACT |
| | GGGACCAAGGTCACCGTCCTA (SEQ ID NO: 80) |
| 2447_ | GAGGTGCAGCTGTTGGAGTCTGGGGGAGGCTTGGTACAGCCTGGCAGG |
| 3H02 | TCCCTGAGACTCTCCTGTGCAGCCTCTGGATTCACCTTTGATGATTATG |
| | CCATGCACTGGGTCCGGCAAGCTCCAGGGAAGGGCCTGGAGTGGGTCT |
| | CAGGTATTAGTTGGGATAGTGGTAGCATAGGCTATGCGGACTCTGTGA |
| | AGGGCCGATTCACCATCTCCAGAGACAACGCCAAGAACTCCCTGTATC |
| | TGCAAATGAACAGTCTGAGAGCTGAGGACACGGCCTTGTATTACTGTG |
| | CAAAAGATGTTAGCAGTGGCTGGTACTGGTATGCTTTTGATATCTGGG |
| | GCCAGGGAACCCTGGTCACCGTCTCGAGTGGTGGAGGCGGTTCAGGCG |
| | GAGGTGGCTCTGGCGGTGGCGCTAGCTCCTATGAGCTGACTCAGGACC |
| | CTGCTGTGTCTGTGGCCTTGGGACAGACAGTCAGGATCACATGCCAAG |
| | GAGACATCCTCAGAAGTTATTATGCAAGTTGGTACCAGCAGAAGCCAG |
| | GACAGGCCCCTGTACTTGTCATTTATGGTAAAAACAACCGGCCCTCAG |
| | GGATCCCAGACCGATTCTCTGGCTCCAGCTCAGGAAACACAGCTTCCT |
| | TGACCATCACTGGGGCTCAGGCGGAAGATGAGGCTGACTATTACTGTA |
| | ACTCCCGGGACAGCAGTGGTAACCGTGTGGTATTCGGCGGAGGGACCA |
| | AGCTGACCGTCCTA (SEQ ID NO: 90) |
| 2447_ | GAGGTGCAGCTGTTGGAGTCTGGGGGAGGCTTGGTACAGCCTGGCAGG |
| 3H08 | TCCCTGAGACTCTCCTGTGCAGCCTCTGGATTCACCTTTGATGATTATG |
| | CCATGCACTGGGTCCGGCAAGCTCCAGGGAAGGGCCTGGAGTGGGTCT |
| | CAGGTATTAGTTGGAATAGTGGTAGCATAGACTATGCAGACTCCGTGA |
| | AGGGCCGATTCACCATCTCCAGAGACAATTCCAAGAACACGCTGTATC |
| | TGCAAATGAACAGCCTGAGAGCTGAGGACACAGCTGTGTACTACTGTG |
| | CGAGAGATCGGAGTAGTGGCTGGTACACGGGGTCCTTTGACTACTGGG |
| | GCCAGGGCACCCTGGTCACCGTCTCGAGTGGTGGAGGCGGTTCAGGCG |
| | GAGGTGGCTCTGGCGGTGGCGCTAGCTCCTATGAGCTGACTCAGGACC |
| | CTGCTGTGTCTGTGGCCTTGGGACAGACAGTCAGGATCACATGCCAAG |
| | GAGACAGCCTCAGAAGCTATTATGCAAGCTGGTACCGGCAGAAGCCA |
| | GGACAGGCCCCTGTACTTGTCATCTATGGTAAAAACAACCGGCCCTCA |
| | GGGATCCCAGACCGATTCTCTGGCTCCAGCTCGGGAAACACAGCTTCC |
| | TTGACCATCATTGGGGCTCAGGCGGAAGACGAGGCTGACTATTACTGT |
| | CAGTCCCGGGACAGCAGTGATAACCGTGTTCTATTCGGCGGAGGGACC |
| | AAGGTCACCGTCCTA (SEQ ID NO: 100) |

Affinity of scFv-Fc antibody including the scFv for mucin1 antigen was measured using Biacore T200 (GE healthcare). Anti-human IgG (Fc) antibody (GE healthcare, Cat. No. BR-1008-39, final concentration of 25 ug/mL) was applied at a rate of 5 uL/min for 360 seconds on a Series S Sensor Chip CM5 (GE healthcare, Cat. No. BR-1005-30) using an Amine Coupling Kit (GE healthcare, Cat. No. BR-1000-50) and immobilized at about 5000-7000 RU. Human MUC1 protein as the antigen was injected at a rate of 30 μL/min at different five concentrations from 25 nM to 400 nM, and kₐ and k_{d} values were obtained as in the following Table, and K_{D} values were calculated therefrom. As a result, K_{D} (M) values indicating binding affinity for mucin1 are shown in Table 5.

TABLE 5

| Clone name | K_{D} (M) |
|---|---|
| 2447_2H08 | 94.8 × 10⁻⁹ |
| 2447_3A01 | 51.5 × 10⁻⁹ |
| 2447_3A08 | 320.3 × 10⁻⁹ |
| 2447_3A09 | 62.1 × 10⁻⁹ |
| 2447_3A12 | 13.8 × 10⁻⁹ |
| 2447_3B07 | 51.7 × 10⁻⁹ |
| 2447_3C08 | 24.0 × 10⁻⁹ |
| 2447_3H02 | 58.8 × 10⁻⁹ |
| 2447_3H08 | 39.9 × 10⁻⁹ |
| G03 | 27.6 × 10⁻⁹ |

### Example 2. Preparation of CAR-T Cells Using scFv Binding to Mucin1

### 2-1. Preparation of CAR-Expressing Lentivirus Transfer Vector

A lentivirus vector (transfer vector or expression vector) expressing a chimeric antigen receptor (CAR) comprising an extracellular domain comprising the scFv polypeptide sequence (a nucleotide sequence of Table 4), a hinge (CD8a) (SEQ ID NO: 112), and a transmembrane domain (CD28) (SEQ ID NO: 113), a co-stimulatory domain (CD28) (SEQ ID NO: 114), and an intracellular signaling domain (CD3 zeta) (SEQ ID NO: 115) was prepared. The scFv region of CAR was isolated by PCR from a plasmid supplied by the screening company, and the hinge, transmembrane protein, and CD28 and CD3z signaling domains were isolated from the synthesized plasmid by PCR. Two fragments were cloned into a Clonetech's pLVX-EF1alpha vector using a Takara's In-Fusion HD cloning kit. The lentivirus vector thus cloned was transfected into Takara's Lenti-X 293T cell line (product code 632180) together with a packaging plasmid provided by the same company's Lenti-X Expression System (EF1a Version) (product code 631253), and 24 hours later, the soup was separated and the lentivirus therein was used for further research.

### 2-2. Production of Lentivirus Particles

To produce the lentivirus, the lentivirus transfer vector (transfer vector or expression vector) prepared above and a packaging plasmid mix were transfected into Lenti-X 293T cells (Takara product no. 632180) using a liposome nucleic acid transfection reagent (lipofectamine, Gibco). One day before transfection, HEK293T cells were prepared at confluency of about 6% to 70% of a culture plate area. Dulbecco's modified Eagle medium (DMEM, Gibco) supplemented with 10% heat-inactivated fetal bovine serum (FBS, Gibco) was used as a medium, and a culture plate of 100 mm or 150 mm in size was used. Culture conditions were maintained at 37° C., 5% CO₂, and humidified conditions. The transfer vector, the lentivirus packaging plasmid mix, and the liposome nucleic acid transfection reagent were mixed at room temperature by adjusting their ratio according to the manufacturer's instructions, and dropped on the HEK293T cells on the plate during culture to transfect the HEK293T cells. After transfection, the lentiviruses produced in the HEK293T cells were burst out of the cells and was present in the form of virus particles in the cell culture. 48 hours after transfection, only the cell culture solution was removed from the culture plate and centrifuged to obtain lentivirus particles in the form of pellet. The pellet was suspended using a solution, which was then immediately used or stored in a cryogenic freezer. As the solution, a DMEM medium used above, or a solution was used, which was prepared by adding Optimizer T-Cell Expansion Supplement (26 ml) to a 1 L bottle of CTS optimizer T cell Expansion SFM (Gibco) used in T cell culture, adding CTS Immune Cell SR (Serum Replacement, Gibo) at a final concentration of 2%, and then adding CTS GlutaMAX-I Supplement 100× (Gibco) at a final concentration of 1×.

### 2-3. Preparation of CAR-T Cell

Peripheral blood mononuclear cell (PBMC) samples were supplied through a research agreement with the Catholic University of Korea, and donor recruitment and blood collection were carried out with the approval of the Catholic University of Korea-Institutional Review Board. All blood was of normal people. In exemplary embodiments of the present disclosure, “Donor” refers to the donor of the PBMC sample used in the experiment, and the number after Donor means that the donor is different (for example, indicated by Donor 03, Donor 04, Donor 05, Donor 06, Donor 07, Donor 08, Donor 18-09, Donor 20-01, etc.). To prepare CAR-T cells expressing the chimeric antigen receptor (CAR) on the surface of T cells, frozen PBMC vials were thawed in a water bath at 37° C. The next day after thawing of frozen PBMCs, the number of cells was counted, followed by replacement with a fresh medium. At the same time, T-cells were activated using T cell-specific expansion beads (Dynabeads Human T-expander CD3/CD28, Gibco), and chimeric antigen receptor (CAR) in the form of lentivirus was transduced by treating T cells according to the number of activated cells. In order to culture the transduced T cells, a suspension of interleukin-2 (Norvatis) in filtered sterile distilled water was added at 100 IU/ml, based on the amount of the medium, and during the subsequent culture period, the suspension was continuously added when the medium was replaced or added. The cell culture period was 10 days to 11 days in total after activation, and cell culture was performed while replacing or newly adding the medium every 1-2 days depending on the number of cells. At the end of the culture period, the cell culture was completed and CAR-T cell production was completed. The prepared CAR-T cells were used immediately, or mixed with a cell cryopreservation solution and injected into a vial for cell freezing and stored in a liquid nitrogen tank. The expression levels of the chimeric antigen receptor (CAR) of the prepared CAR-T cells and characteristics thereof were analyzed by flow cytometry (FACS).

### Example 3. Analysis of Affinity of Soluble scFv to Mucin1-Expressing Cell

Hereinafter, unless otherwise specified, information on cell lines used in the present exemplary embodiment is as follows:
PANC-1; Korean Cell Line Bank, 21469
HCC1954; Korean Cell Line Bank, 9S1954
HCC70; Korean Cell Line Bank, 9S0070
T47D; Korean Cell Line Bank, 30133
HEK293T; ATCC, CRL-3216
Huh7; Korean Cell Line Bank, 60104
HepG2; ATCC, HB-8065
SK-N-SH; Korean Cell Line Bank, 30011

A total of 4 cell lines were used in Example 3, and among them, HEK293 (ATCC) and PANC-1 (ATCC) cell lines were used as negative controls, and HEK293T-v1 (ATCC and transformed by LG Chem) and T47D (Korean Cell Line Bank) were used as positive controls. HEK293T-v1 cell line used as the positive control is a cell line obtained by expressing C subunit of MUC1 in HEK293T cell line not expressing mucin1. 11 types of scFvs were used, and among them, a positive control was G03 (Table 3). The soluble scFv prepared in Example 1 was diluted 5-fold to prepare a total of 8 serial dilutions. Each serial dilution was 380 μg/mL, 76 μg/mL, 15.2 μg/mL, 3.04 μg/mL, 0.608 μg/mL, 0.1216 μg/mL, 0.02432 μg/mL, 0.004864 μg/mL. A buffer used in soluble scFv dilution and cell washing was PBS containing 2% FBS and 10 mM EDTA. Four kinds of cells were prepared at a density of 1×10⁶/mL, respectively, and put in a 96 well U-bottom cell culture plate at a density of 1×10⁵/100 μl/well, followed by centrifugation at 1500 RPM for 5 minutes. The soluble scFv prepared previously was treated in an amount of 50 μl/well. The cells were left at 4° C. for 1 hour. 150 μl/well of the cell washing buffer was added thereto and then mixed, followed by centrifugation at 1500 RPM for 5 minutes. The supernatant was discarded, and 200 μl/well of the cell washing buffer was added thereto and then mixed, followed by centrifugation at 1500 RPM for 5 minutes. A goat anti-human IgG Fc-biotin antibody (Invitrogen, A18821) was diluted 500-fold, and 50 μl/well thereof was treated. The cells were left at 4° C. for 20 minutes. 150 μl/well of the cell washing buffer was added thereto and then mixed, followed by centrifugation at 1500 RPM for 5 minutes. The supernatant was discarded, and 200 μl/well of the cell washing buffer was added thereto and then mixed, followed by centrifugation at 1500 RPM for 5 minutes. PE-Streptavidin (BD Pharmigen, 554061) was diluted 1000-fold, and 50 μl/well thereof was treated. The cells were left at 4° C. for 20 minutes. 150 μl/well of the cell washing buffer was added thereto and then mixed, followed by centrifugation at 1500 RPM for 5 minutes. The supernatant was discarded, and 200 μl/well of the cell washing buffer was added thereto and then mixed, followed by centrifugation at 1500 RPM for 5 minutes. 80 μl/well of the cell washing buffer was added and mixed, followed by flow cytometry.

The affinity of the soluble scFv to the mucin1-expressing cells was confirmed by PE MFI values from the results of the flow cytometry. Each cell line without soluble scFv was stained with another fluorescent antibody (Goat anti-human IgG Fc-biotin antibody, PE-Streptavidin), which was used as a negative control value, and each cell line was treated with G03 at a concentration of 380 μg/mL, which was used as a positive control value, and MFI value was corrected as follows:

(Experimental Value−Negative Control Value)/(Positive Control Value−Negative Control Value)×100=Correction value of Experimental Value

EC₅₀ was calculated using the corrected values, and the described results are the results of two cell lines used as positive controls (Table 6).

TABLE 6

| Clone name | EC₅₀ (293T-v1) (ug/ml) | EC₅₀ (T47D) (ug/ml) |
|---|---|---|
| G03 | 3.46 | 3.56 |
| 2445_1F09 | 1.51 × 10⁷ | 6.19 × 10⁷ |
| 2447_3A01 | 4.78 | 11.14 |
| 2447_3A08 | 1.73 | 3.76 |
| 2447_3A09 | 1.96 | 2.2 |
| 2447_3A12 | 1.47 | 0.75 |
| 2447_3C08 | 1.38 | 2.04 |
| 2447_3H02 | 1.41 | 1.6 |
| 2447_3H08 | 2.57 | 5.6 |
| 2447_2H08 | 21.73 | 130.4 |
| 2447_3B07 | 3.86 | 3.47 |

### Example 4. Analysis of Cytokine Secretion of CAR-T Cells

Responses of immune cells (T cells), into which anti MUC1 scFv was injected in the form of CAR, for MUC1-positive tumor cells (T47D, HEK293T-V1) were evaluated. T47D cells naturally expressing high levels of MUC1, and HEK293T-V1 cells overexpressing C subunit of human MUC1 were co-cultured with a total of 10 kinds of immune cells, 2445_1F09, 2447_3A01, 2447_3A08, 2447_3A09, 2447_3A12, 2447_3C08, 2447_3H02, 2447_3H08, 2447_2H08, 2447_3B07 prepared according to Example 2 (in some drawings, abbreviated as 1F09, 3A01, 3A08, 3A09, 3A12, 3C08, 3H02, 3H08, 2H08, 3B07, respectively), into which anti MUC1 scFv was injected in the form of CAR, at a ratio of tumor cell:immune cell of 1:1.5 for 24 hours. Concentrations of two kinds of cytokines, i.e., interferon-gamma (IFNg) and interleukin-2 (IL-2) in the culture media obtained therefrom were examined by ELISA. ELISA was performed in the same manner as the method commonly used, and briefly, as follows. First, a 96-well plate was coated with a capture antibody capable of capturing interferon-gamma or interleukin-2. 1 day later, the plate was sufficiently washed with a washing buffer. A block buffer was added to each well, and left at room temperature for 1 hour, followed by sufficiently washing with the washing buffer. In order to know the absolute value, along with a standard solution of known concentration, a culture solution sample was added to each well, left at room temperature for 2 hours, and allowed to react. The plate was sufficiently washed with the washing buffer, and then a detection antibody was added thereto and allowed to react at room temperature for 2 hours. The plate was sufficiently washed with the washing buffer, and then an HRP-conjugated streptavidin was added to each well, and allowed to react at room temperature for 20 minutes. The plate was sufficiently washed with the washing buffer, and then a substrate solution was added, and allowed to react at room temperature for 20 minutes. The reaction was stopped with a stop solution. The cytokine concentration in each well was calculated by absorbance according to reaction.

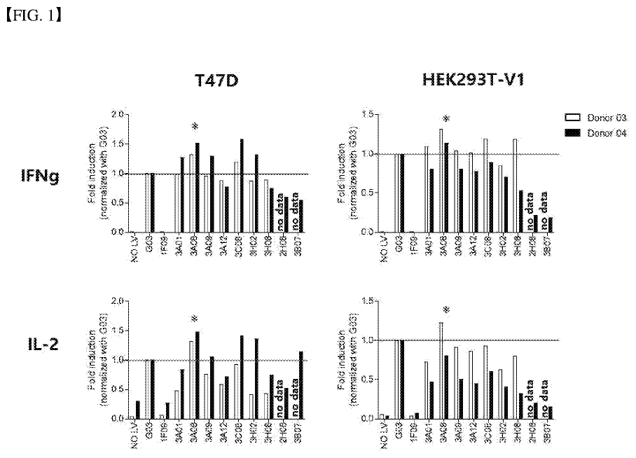

In FIG. 1, G03 indicates a positive control, and as a negative control, a group (NO LV) into which no CAR was injected was used. In addition, FIG. 1 shows representative graphs showing the results of independent experiments using a total of two kinds of T cells, in which they were named Donor 03 and Donor 04 according to the origin of T cells. In the present exemplary embodiment, “Donor” refers to the donor of the T cell used in the experiment, and the number after Donor means that the donor is different. FIG. 1 shows graphs in which the absorbance was normalized, based on the control G03, showing that the tested immune cells secreted cytokines.

FIG. 2 shows results of cytokine secretion analysis evaluating responses of immune cells, into which anti-MUC1 scFv was injected in the form of CAR, for MUC1-positive tumor cells (T47D), showing that the tested immune cells secreted cytokines. T cells were derived from Donor 06. A group (Control T cell) into which no CAR was injected was used as a negative control.

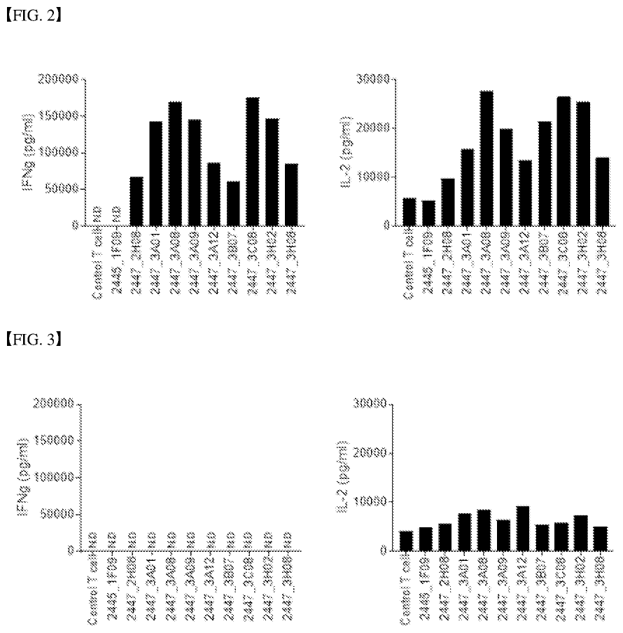

FIG. 3 shows results of cytokine secretion analysis evaluating responses of immune cells, into which anti-MUC1 scFv was injected in the form of CAR, for cells not expressing mucin1 (HEK293T), showing that the immune cells did not secrete interferon gamma cytokine, and the secretion amount of IL-2 did not increase significantly, as compared to that of the negative control (Control T cell). T cells were derived from Donor 06. A group (Control T cell) into which no CAR was injected was used as the negative control.

### Example 5. Analysis of Cytotoxicity of CAR-T Cell

Human breast cancer cell lines (T47D, HCC70, HCC1954), human liver cancer cell lines (Huh7, HepG2), and a human neuroblastoma cell line (SK-N-SH) were purchased from Korean Cell Line Bank (KCLB, Seoul, Korea), and a human embryonic kidney cell line (HEK293T) was purchased from American Type Culture Collection (ATCC, Manassas, VA). T47D, HCC70, and HCC1954 cells were cultured in an RPMI-1640 medium (Thermo Fisher Scientific), and HEK293T, Huh7, and HepG2 were cultured in a Dulbecco's modified Eagle's medium (DMEM, Thermo Fisher Scientific), respectively. All media were used after supplemented with 10% (w/v) heat inactivated fetal bovine serum (FBS, Thermo Fisher Scientific). All cells were cultured under conditions of 37° C. and 5% CO₂. CAR-T cells prepared according to Example 2 were cultured in a CTS complete medium (CTS™ OpTmizer™ T Cell Expansion SFM (Gibco, A1048501), 2% (w/v) CTS™ Immune Cell SR (Gibco, A25961-01), 1× GlutaMAX™ Supplement (Gibco, 35050-061)) under conditions of 37° C. and 5% CO₂.

To analyze mucin1-specific cytotoxicity of CAR-T cells, cell lysis was measured for mucin1-expressing cells (including T47D, HCC70, HCC1954, HEK293T-V1 (exogenous MUC1-C-expressing cell), etc.) and cells not expressing mucin1 (including HEK293T, HepG2, Huh7, SK-N-SH, etc.) by the following method. First, target cells expressing or not expressing mucin1, which were stained with CellTrace™ CFSE (Invitrogen, C34554), were dispensed at a density of 0.15˜0.2×10⁶ cells/150 μl/well, and cultured for one day. On the same day, CAR-T cells frozen and stored at −196° C. were thawed, and cultured at a density of 2×10⁶ cells/ml for one day in a CTS complete medium containing 30 U/ml of IL-2 (Novartis, Proleukin). The next day, the CAR expression rate of CAR-T cells during culture was measured by staining with Alexa Fluor 488-AffiniPure Goat Anti-Human IgG, F(ab′)2 Fragment Specific (Jackson laboratory, 109-545-097) and APC Mouse Anti-Human CD3 (BD Pharmingen™, 561811), and obtaining the percentage of cells positive for both of them using a flow cytometer (FACS). Each type of CAR-T (E, effector cells), adjusted to have the same number of CAR+ T cells, based on the CAR expression rate, was prepared in a volume of 100 μl/well using a cell culture medium of each target cell, and added in proportion to the number of CFSE-stained cells expressing or not expressing mucin1 (T, target cells) cultured the day before. The cells were cultured under conditions of 37° C., 5% CO₂ for 5 hours up to 24 hours depending on the target cells. Thereafter, the CAR-T cells and target cells were all taken, and stained with Fixable Viability Dye eFluor™ 450 (eBioscience™, 65-0863-18), APC Mouse Anti-Human CD3. Then, the number of live target cells was obtained using a flow cytometer. In this regard, the number of live target cells was determined by Fixable Viability Dye-negative, CFSE-positive cells. Killing activity (%) was calculated by the following equation:

$$Killing activity = 100 - ( the number of live Target cells in ( T cell + Target cell ) well / the number of live Target cells in ( Target cell ) well \times 100 ) ( \% )$$

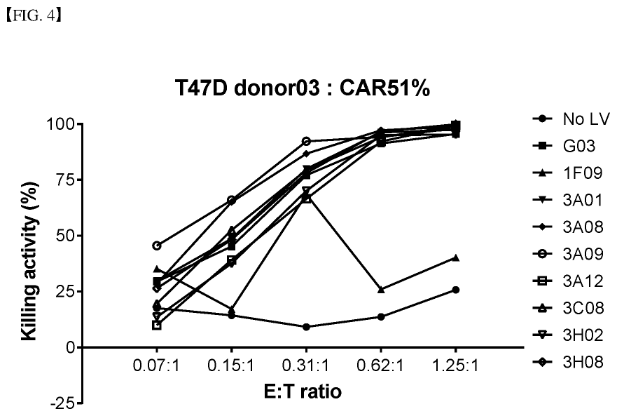

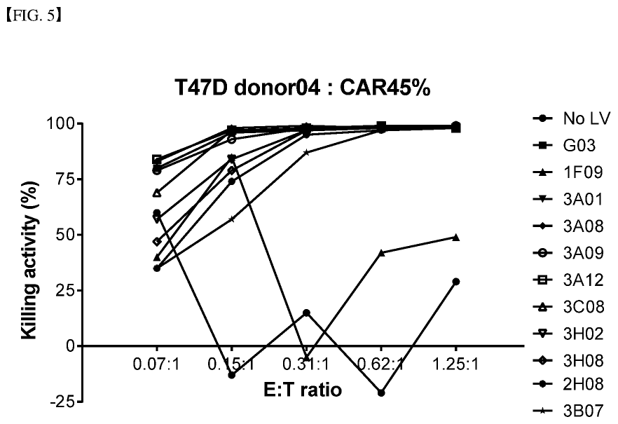

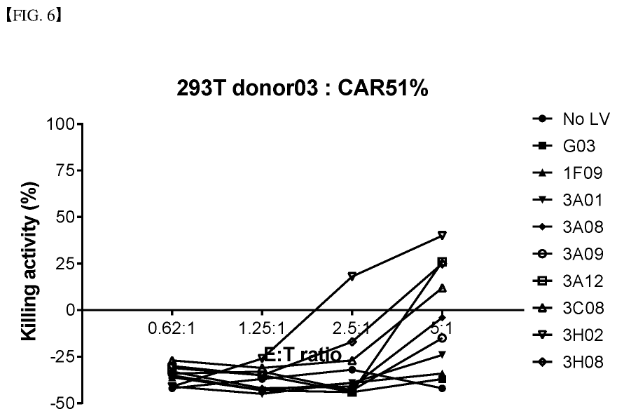

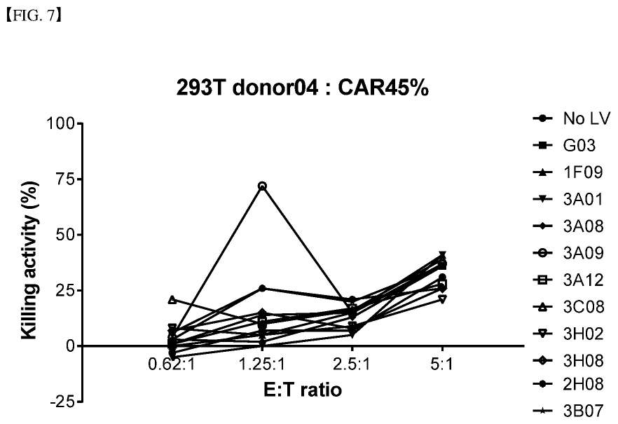

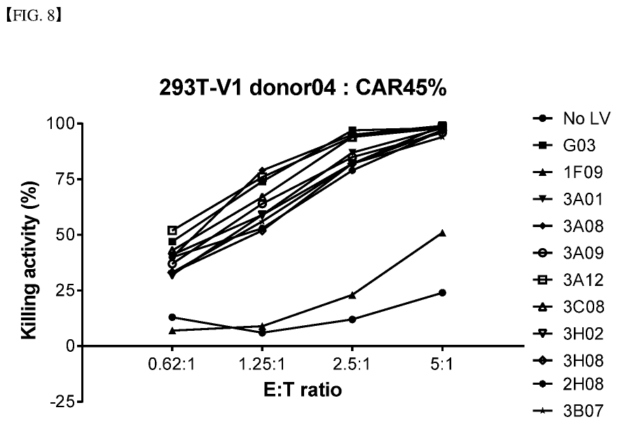

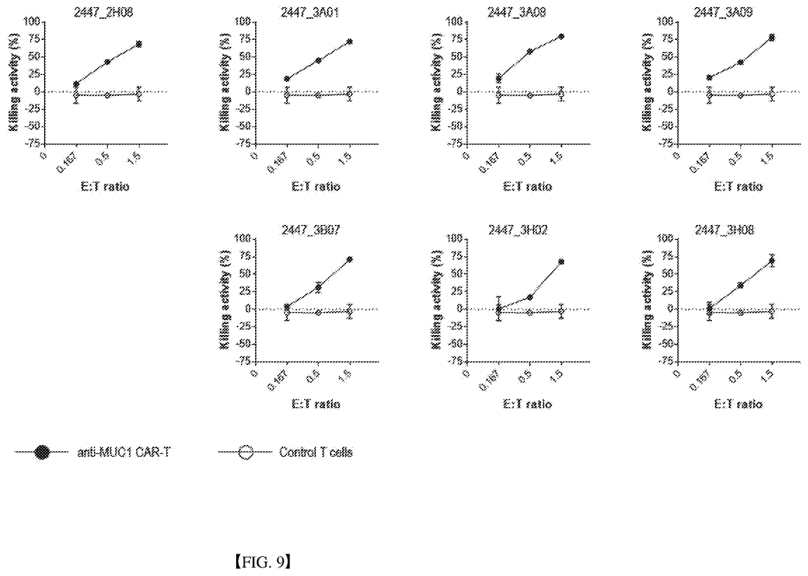

The results are shown in FIGS. 4 to 8. In the drawings, G03 represents a positive control, and as a negative control, a group (NO LV) into which no CAR was injected was used. Further, these drawings show representative graphs showing the results of independent experiments using a total of two kinds of T cells, in which they were named Donor 03 and Donor 04 according to the origin of T cells. As shown in FIGS. 4 to 8, the tested immune cells exhibited cytotoxicity against mucin1-expressing cells.

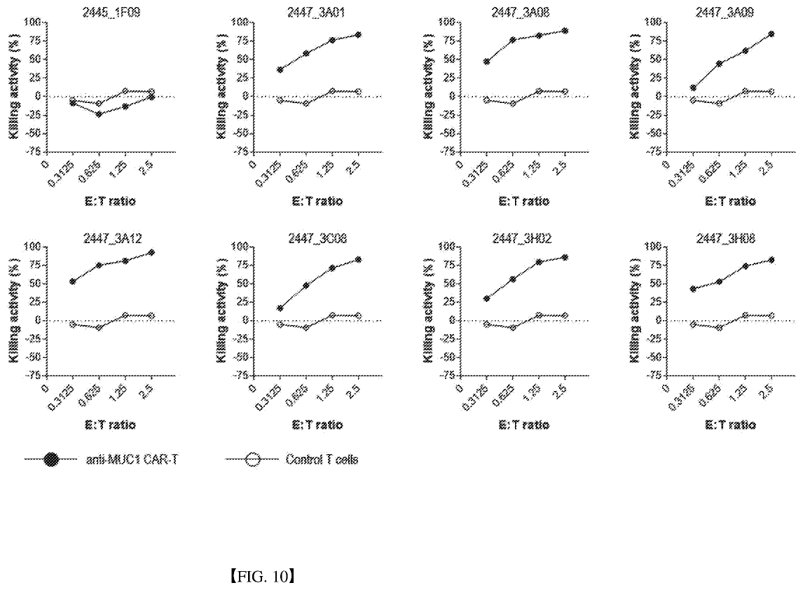

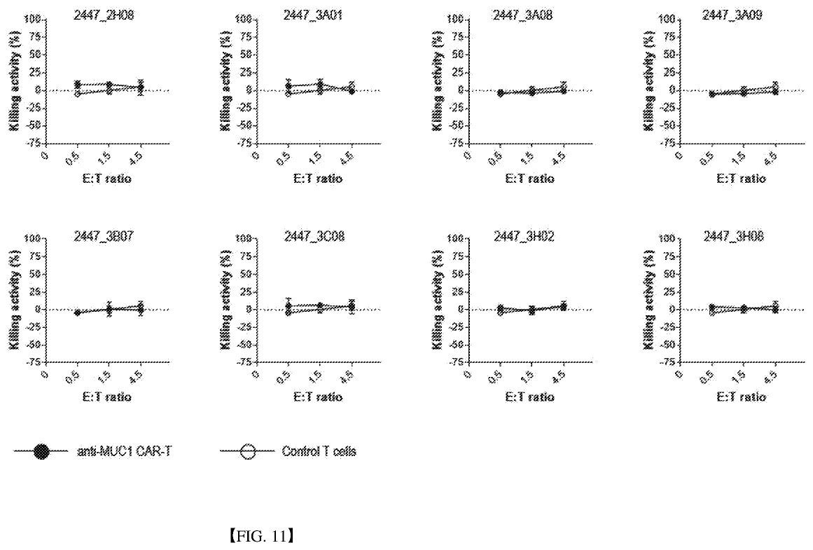

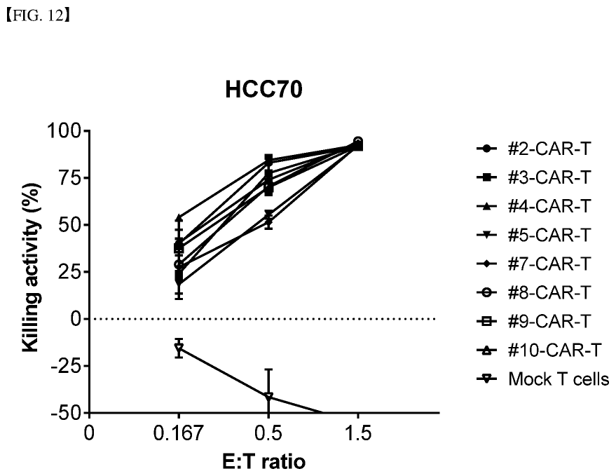

FIGS. 9 and 10 show experimental results of cytotoxicity of CAR-T cells prepared by using Donor 05- and Donor 06-derived T cells against a human breast cancer cell line T47D, FIG. 11 shows experimental results of cytotoxicity of CAR-T cells prepared by using Donor 07-derived T cells against a human embryonic kidney cell line HEK293T, and FIG. 12 shows experimental results of cytotoxicity of CAR-T cells prepared by using Donor 08-derived T cells against a human breast cancer cell line HCC70. As shown in FIGS. 9 to 12, the tested immune cells exhibited cytotoxicity against mucin1-expressing cells.

### Example 6. Analysis of Mucin1-Specific Cytotoxicity of CAR-T Cell

To examine whether CAR-T cells are able to differentiate normal cells from cancer cells according to the difference in the amount of antigen, cell lysis of a breast cancer cell line (HCC70) and a human normal breast cell line (MCF10A) was measured as follows: First, HCC70 cells overexpressing mucin1 and MCF10A cells expressing low levels of mucin1 were stained with different concentrations of CellTrace™ CFSE (Invitrogen, C34554), and mixed at different ratios, respectively. The ratios of HCC70: MCF10A were 0:0.2, 0.025:0.175, 0.05:0.15, 0.075:0.125, 0.1:0.1, 0.125:0.075, 0.15:0.05, 0.175:0025, 0.2:0. The cells mixed at the predetermined ratios were dispensed at a density of 0.2×10⁶ cells/500 μl/well, and cultured for one day. On the same day, CAR-T cells (Donor 05-derived T cells expressing CAR containing 2447_2H08 scFv) frozen and stored at −196° C. were thawed, and cultured at a density of 2×10⁶ cells/ml for one day in a CTS complete medium containing 30 U/ml of IL-2 (Novartis, Proleukin). The next day, the CAR (CAR containing 2447_2H08 scFv) expression rate of CAR-T cells during culture was measured by staining with Alexa Fluor 488-AffiniPure Goat Anti-Human IgG, F(ab′)2 Fragment Specific (Jackson laboratory, 109-545-097) and APC Mouse Anti-Human CD3 (BD Pharmingen™, 561811), and obtaining the percentage of cells positive for both of them using a flow cytometer (FACS). Each type of CAR-T (E, effector cells), adjusted to have the same number of CAR+ T cells, based on the CAR expression rate, was prepared in a volume of 100 μl/well using a cell culture medium of each target cell, and added in proportion to the number of CFSE-stained cells expressing or not expressing mucin1 (T, target cells) cultured the day before. The cells were cultured under conditions of 37° C., 5% CO₂ for 24 hours in the target cells. Thereafter, the CAR-T cells and target cells were all taken, and stained with Fixable Viability Dye eFluor™ 450 (eBioscience™, 65-0863-18), APC Mouse Anti-Human CD3. Then, the number of live target cells was obtained using a flow cytometer. In this regard, the number of live target cells was determined by Fixable Viability Dye-negative, CFSE-positive cells (HCC70 by strong CFSE signal and MCF10A by weak CFSE signal). Killing activity (%) was calculated in the same manner as in Example 5.

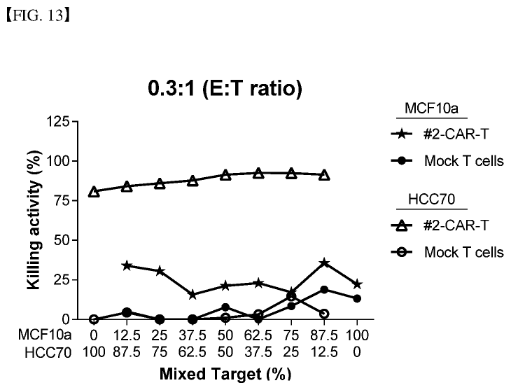

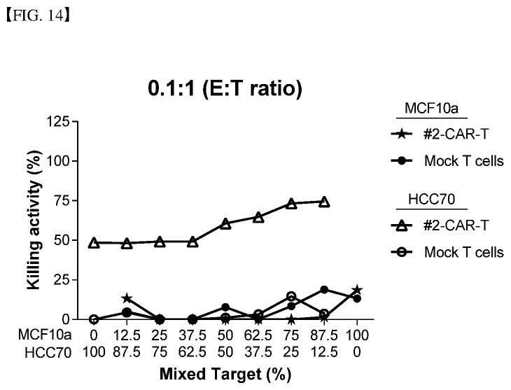

The results are shown in FIGS. 13 and 14. In the drawings, as a negative control, a group (mock T cell) into which no CAR was injected was used. As shown in FIGS. 13 to 14, CAR-T cells having 2447_2H08 scFv (in the drawings, each indicated by #2-CAR-T) exhibited cell killing activity against the breast cancer cell line (HCC70) overexpressing mucin1, whereas they exhibited no cytotoxicity or remarkably reduced cytotoxicity against the normal breast cell line (MCF10A) expressing low levels of mucin1.

### Example 7. Analysis of Anticancer Efficacy of CAR-T Cell Using Animal Model

7-1. Analysis of anticancer efficacy using pancreatic cancer animal model 7-week-old NOG female mice were subcutaneously (s.c.) injected with 6×10⁶ PANC1-v1 cells (PANC1 pancreatic cancer cells expressing MUC1). When the tumor volume reached 500 mm³, 1×10⁶ anti-MUC1 CAR-T cells (Donor 18-09-derived T cells expressing scFv-containing CAR according to the present disclosure) were intravenously (i.v.) injected, and then tumor growth was observed. 14 days and 28 days after CAR-T cell injection, blood was collected, and the number of human immune cells (hCD45+ cells) in the blood was observed (n=5 per group). As negative controls, a PBS (Vehicle)-injected group, and T cells not expressing CAR (Control T cell) were used.

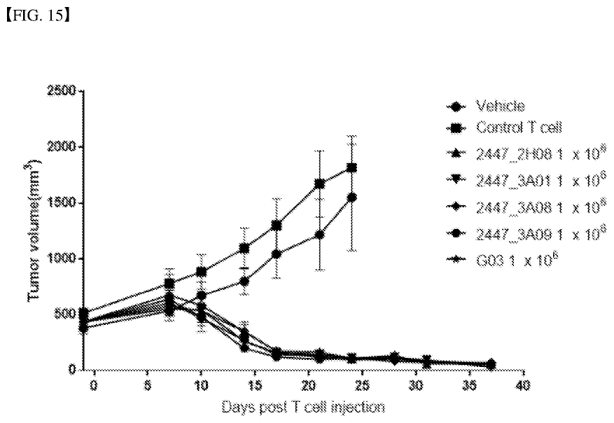

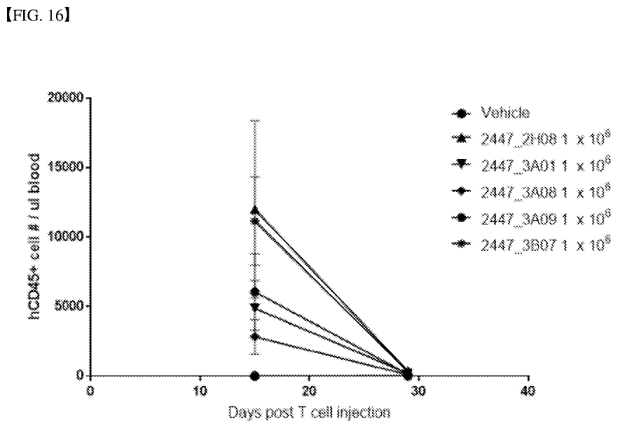

As a result, as shown in FIGS. 15 and 16, it was observed that tumor of 500 mm³ in size became small, and about 20 days after T cell injection, no tumor was observed in appearance. In contrast, in the negative controls, the tumor size continued to increase.

Next, in the same animal model, when the tumor volume exceeded 1500 mm³ (i.e., 66 days after PANC1-v1 cell injection), 1×10⁶ or 0.5×10⁶ anti-MUC1 CAR-T cells were intravenously (i.v.) injected, and then tumor growth was observed (n=2 per group).

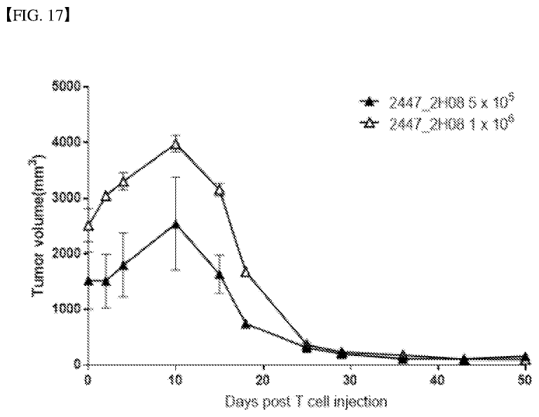

As a result, as shown in FIG. 17, the tumor size increased in the early stage. However, after about 10 days, the size gradually decreased, and after 30 days, the tumor size became too small to measure.

### 7-2. Analysis of Anticancer Efficacy Using Breast Cancer Animal Model

9-week-old NOG female mice were subcutaneously (s.c.) injected with 4×10⁶ HCC1954 cells (breast cancer cell line naturally expressing MUC1). When the tumor volume reached 500 mm³ (23 days after cell injection), 0.5×10⁶ or 0.1×10⁶ anti-MUC1 CAR-T cells (Donor 20-01-derived T cells expressing scFv-containing CAR according to the present disclosure) were intravenously (i.v.) injected, and then tumor growth was observed (n=7 per group). After T cell injection, blood was collected at regular intervals, and the number of human immune cells (hCD45+ cells) in the blood was observed (n=3 per group). As negative controls, a PBS (Vehicle)-injected group, and T cells not expressing CAR (Control T cell) were used.

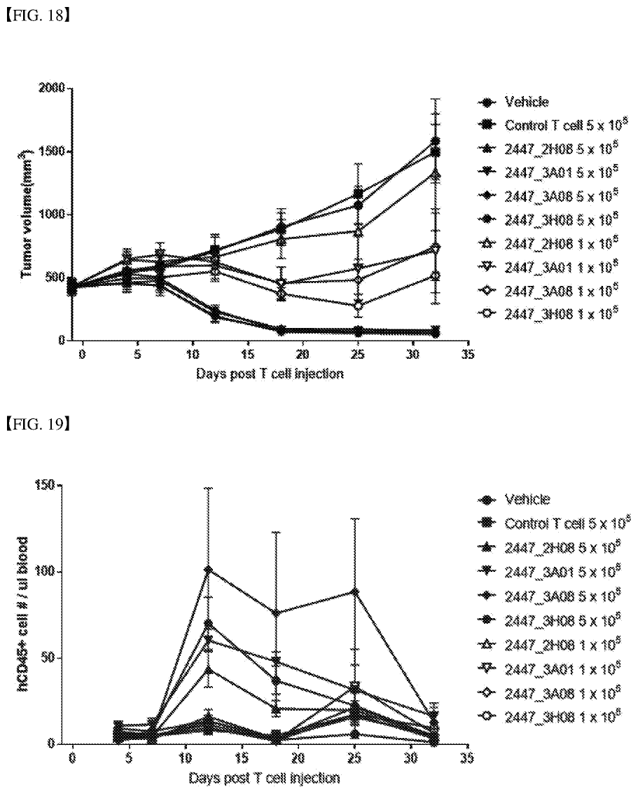

As a result, as shown in FIGS. 18 and 19, the tumor of 500 mm³ in size remained stagnant for 7 days after CAR-T cell injection. Thereafter, the size rapidly decreased, and became too small to measure after 25 days. In addition, the number of hCD45+ cells in the mouse blood tended to increase, and the number gradually decreased after about 25 days. It seems that CAR-T cells were activated and proliferated after recognizing the antigen, and thus the number of cells in the blood increased.

Based on the above description, it will be understood by those skilled in the art that the present disclosure may be implemented in a different specific form without changing the technical spirit or essential characteristics thereof. In this regard, it should be understood that the above embodiment is not limitative, but illustrative in all aspects. The scope of the disclosure is defined by the appended claims rather than by the description preceding them, and therefore all changes and modifications that fall within metes and bounds of the claims, or equivalents of such metes and bounds are therefore intended to be embraced by the claims.

1. A polypeptide binding to mucin1, the polypeptide comprising a pair of a heavy chain variable (VH) region and a light chain variable (VL) region selected from the following VH and VL regions:
a VH region comprising complementarity-determining region (CDR) 1, CDR 2, and CDR 3 represented by amino acid sequences of SEQ ID NO: 1, SEQ ID NO: 2, and SEQ ID NO: 3, respectively, and a VL region comprising CDR 1, CDR 2, and CDR 3 represented by amino acid sequences of SEQ ID NO: 4, SEQ ID NO: 5, and SEQ ID NO: 6, respectively;
a VH region comprising CDR 1, CDR 2, and CDR 3 represented by amino acid sequences of SEQ ID NO: 11, SEQ ID NO: 12, and SEQ ID NO: 13, respectively, and a VL region comprising CDR 1, CDR 2, and CDR 3 represented by amino acid sequences of SEQ ID NO: 14, SEQ ID NO: 15, and SEQ ID NO: 16, respectively;
a VH region comprising CDR 1, CDR 2, and CDR 3 represented by amino acid sequences of SEQ ID NO: 21, SEQ ID NO: 22, and SEQ ID NO: 23, respectively, and a VL region comprising CDR 1, CDR 2, and CDR 3 represented by amino acid sequences of SEQ ID NO: 24, SEQ ID NO: 25, and SEQ ID NO: 26, respectively;
a VH region comprising CDR 1, CDR 2, and CDR 3 represented by amino acid sequences of SEQ ID NO: 31, SEQ ID NO: 32, and SEQ ID NO: 33, respectively, and a VL region comprising CDR 1, CDR 2, and CDR 3 represented by amino acid sequences of SEQ ID NO: 34, SEQ ID NO: 35, and SEQ ID NO: 36, respectively;
a VH region comprising CDR 1, CDR 2, and CDR 3 represented by amino acid sequences of SEQ ID NO: 41, SEQ ID NO: 42, and SEQ ID NO: 43, respectively, and a VL region comprising CDR 1, CDR 2, and CDR 3 represented by amino acid sequences of SEQ ID NO: 44, SEQ ID NO: 45, and SEQ ID NO: 46, respectively;
a VH region comprising CDR 1, CDR 2, and CDR 3 represented by amino acid sequences of SEQ ID NO: 51, SEQ ID NO: 52, and SEQ ID NO: 53, respectively, and a VL region comprising CDR 1, CDR 2, and CDR 3 represented by amino acid sequences of SEQ ID NO: 54, SEQ ID NO: 55, and SEQ ID NO: 56, respectively;
a VH region comprising CDR 1, CDR 2, and CDR 3 represented by amino acid sequences of SEQ ID NO: 61, SEQ ID NO: 62, and SEQ ID NO: 63, respectively, and a VL region comprising CDR 1, CDR 2, and CDR 3 represented by amino acid sequences of SEQ ID NO: 64, SEQ ID NO: 65, and SEQ ID NO: 66, respectively;
a VH region comprising CDR 1, CDR 2, and CDR 3 represented by amino acid sequences of SEQ ID NO: 71, SEQ ID NO: 72, and SEQ ID NO: 73, respectively, and a VL region comprising CDR 1, CDR 2, and CDR 3 represented by amino acid sequences of SEQ ID NO: 74, SEQ ID NO: 75, and SEQ ID NO: 76, respectively;
a VH region comprising CDR 1, CDR 2, and CDR 3 represented by amino acid sequences of SEQ ID NO: 81, SEQ ID NO: 82, and SEQ ID NO: 83, respectively, and a VL region comprising CDR 1, CDR 2, and CDR 3 represented by amino acid sequences of SEQ ID NO: 84, SEQ ID NO: 85, and SEQ ID NO: 86, respectively; and
a VH region comprising CDR 1, CDR 2, and CDR 3 represented by amino acid sequences of SEQ ID NO: 91, SEQ ID NO: 92, and SEQ ID NO: 93, respectively, and a VL region comprising CDR 1, CDR 2, and CDR 3 represented by amino acid sequences of SEQ ID NO: 94, SEQ ID NO: 95, and SEQ ID NO: 96, respectively.

2. The polypeptide of claim 1, comprising a pair of a heavy chain variable (VH) region and a light chain variable (VL) region selected from the following VH and VL regions:
a VH region comprising an amino acid sequence of SEQ ID NO: 7 and a VL region comprising an amino acid sequence of SEQ ID NO: 8;
a VH region comprising an amino acid sequence of SEQ ID NO: 17 and a VL region comprising an amino acid sequence of SEQ ID NO: 18;
a VH region comprising an amino acid sequence of SEQ ID NO: 27 and a VL region comprising an amino acid sequence of SEQ ID NO: 28;
a VH region comprising an amino acid sequence of SEQ ID NO: 37 and a VL region comprising an amino acid sequence of SEQ ID NO: 38;
a VH region comprising an amino acid sequence of SEQ ID NO: 47 and a VL region comprising an amino acid sequence of SEQ ID NO: 48;
a VH region comprising an amino acid sequence of SEQ ID NO: 57 and a VL region comprising an amino acid sequence of SEQ ID NO: 58;
a VH region comprising an amino acid sequence of SEQ ID NO: 67 and a VL region comprising an amino acid sequence of SEQ ID NO: 68;
a VH region comprising an amino acid sequence of SEQ ID NO: 77 and a VL region comprising an amino acid sequence of SEQ ID NO: 78;
a VH region comprising an amino acid sequence of SEQ ID NO: 87 and a VL region comprising an amino acid sequence of SEQ ID NO: 88; and
a VH region comprising an amino acid sequence of SEQ ID NO: 97 and a VL region comprising an amino acid sequence of SEQ ID NO: 98.

3. The polypeptide of claim 1, wherein the polypeptide is an antibody or an antigen-binding fragment thereof.

4. The polypeptide of claim 1, wherein the polypeptide is a single chain variable fragment (scFv), a peptibody, a fragment antigen binding (Fab), a monoclonal antibody, a bispecific antibody, a minibody, a domain antibody, a synthetic antibody, a chimeric antibody, a humanized antibody, a human antibody, or an antibody fusion protein.

5. The polypeptide of claim 1, wherein the VH region and the VL region are linked to each other via a linker.

6. The polypeptide of claim 5, wherein an amino acid sequence of the linker is GGGGSGGGGSGGGAS.

7. The polypeptide of claim 1, wherein the polypeptide is a single chain variable fragment (scFv) and the scFv includes an amino acid sequence selected from the group consisting of SEQ ID NO: 9, SEQ ID NO: 19, SEQ ID NO: 29, SEQ ID NO: 39, SEQ ID NO: 49, SEQ ID NO: 59, SEQ ID NO: 69, SEQ ID NO: 79, SEQ ID NO: 89, and SEQ ID NO: 99.

8. An isolated polynucleotide encoding the polypeptide binding mucin1 of claim 1.

9. A vector comprising the polynucleotide of claim 8.

10. A cell comprising the vector of claim 9.

11. A chimeric antigen receptor comprising the polypeptide binding mucin1 of claim 1.

12. The chimeric antigen receptor of claim 11, comprising an extracellular domain comprising the polypeptide binding mucin1; a transmembrane domain; and an intracellular signaling domain.

13. The chimeric antigen receptor of claim 12, wherein the extracellular domain further includes a spacer region between the polypeptide binding mucin1 and the transmembrane domain.

14. The chimeric antigen receptor of claim 13, wherein the spacer region includes a hinge region of CD8α or CD28, or all or part of a constant region of an immunoglobulin (IgG).

15. The chimeric antigen receptor of claim 12, wherein the transmembrane domain is a transmembrane domain of CD28 or CD8.

16. The chimeric antigen receptor of claim 12, wherein the intracellular signaling domain is a CD3 zeta signaling domain.

17. The chimeric antigen receptor of claim 12, further comprising at least one co-stimulatory domains.

18. The chimeric antigen receptor of claim 17, wherein the at least one co-stimulatory domain is placed between the transmembrane domain and the intracellular signaling domain.

19. The chimeric antigen receptor of claim 17, wherein the at least co-stimulatory domain is a signaling domain of CD28, OX-40, 4-1BB (CD137), CD27, or ICOS.

20. An isolated polynucleotide encoding the chimeric antigen receptor of claim 11.

21. A vector comprising the polynucleotide of claim 20.

22. An immune cell expressing a chimeric antigen receptor comprising the polypeptide binding mucin1 of claim 1, or comprising a polynucleotide encoding the chimeric antigen receptor.

23. The immune cell of claim 22, wherein the immune cell is a T cell, a tumor infiltrating lymphocyte (TIL), a natural killer (NK) cell, a TCR-expressing cell, a dendritic cell, or an NK-T cell.

24. The immune cell of claim 22, wherein the immune cell is an autologous T cell or an allogenic T cell.

25. A composition for preventing or treating cancer, the composition comprising the polypeptide binding mucin1 of claim 1;
an isolated polynucleotide encoding the polypeptide binding mucin1;
a vector comprising the polynucleotide encoding the polypeptide binding mucin1;
a cell comprising the polynucleotide encoding the polypeptide binding mucin1;
a chimeric antigen receptor comprising the polypeptide binding mucin1;
an isolated polynucleotide encoding the chimeric antigen receptor;
a vector comprising the polynucleotide encoding the chimeric antigen receptor; or
an immune cell comprising the polynucleotide encoding the chimeric antigen receptor, or expressing the chimeric antigen receptor.


SEQUENCE LISTING (115 sequences)

SEQ ID NO: 1 - protein, 8 aa, Artificial Sequence
  2445_1F09 HCDR1
GFTVSSNY

SEQ ID NO: 2 - protein, 7 aa, Artificial Sequence
  2445_1F09 HCDR2
IYSGGST

SEQ ID NO: 3 - protein, 10 aa, Artificial Sequence
  2445_1F09 HCDR3
DRGWNHGMDV

SEQ ID NO: 4 - protein, 8 aa, Artificial Sequence
  2445_1F09 LCDR1
SSEVGSRY

SEQ ID NO: 5 - protein, 3 aa, Artificial Sequence
  2445_1F09 LCDR2
KND

SEQ ID NO: 6 - protein, 11 aa, Artificial Sequence
  2445_1F09 LCDR3
AAWDDSLNGYV

SEQ ID NO: 7 - protein, 118 aa, Artificial Sequence
  2445_1F09 VH
EVQLLETGGGLIQPGGSLRLSCAASGFTVSSNYMSWVRQAPGKGLEWVSVIYSGGSTYYADSVKGRFTISRDNSKNTLYLQMNSLRAEDTAVYYCARDRGWNHGMDVWGQGTLVTVSS

SEQ ID NO: 8 - protein, 110 aa, Artificial Sequence
  2445_1F09 VL
QSGLTQPPSASGTPGQRVTISCSGGSSEVGSRYVSWYQQLPGTAPRLLIYKNDRRPSGVPDRFSGSKSGSSASLAISGLRSEDEADYYCAAWDDSLNGYVFGTGTKLTVL

SEQ ID NO: 9 - protein, 243 aa, Artificial Sequence
  2445_1F09 scFv
EVQLLETGGGLIQPGGSLRLSCAASGFTVSSNYMSWVRQAPGKGLEWVSVIYSGGSTYYADSVKGRFTISRDNSKNTLYLQMNSLRAEDTAVYYCARDRGWNHGMDVWGQGTLVTVSSGGGGSGGGGSGGGASQSGLTQPPSASGTPGQRVTISCSGGSSEVGSRYVSWYQQLPGTAPRLLIYKNDRRPSGVPDRFSGSKSGSSASLAISGLRSEDEADYYCAAWDDSLNGYVFGTGTKLTVL

SEQ ID NO: 10 - DNA, 729 bp, Artificial Sequence
  2445_1F09 scFv
gaggtgcagctgttggagactggaggaggcttgatccagcctggggggtccctgagactctcctgtgcagcctctgggttcaccgtcagtagcaactacatgagctgggtccgccaggctccagggaaggggctggagtgggtctcagttatttatagcggtggtagcacatattacgcagactccgtgaagggccgattcaccatctccagagacaattccaagaacacgctgtatcttcaaatgaacagcctgagagccgaggacacggccgtgtattactgtgcgagagatcgcggctggaaccacggtatggacgtctggggccagggaaccctggtcaccgtctcgagtggtggaggcggttcaggcggaggtggctctggcggtggcgctagccagtctgggctgactcagccaccctcagcgtctgggacccccgggcagagggtcaccatctcttgttctgggggcagctcagaagtcggaagtagatatgtgtcctggtaccagcaactcccaggaacggcccccagactcctcatctataagaatgatcggcggccctcaggggtccctgaccgattctctggctccaagtctggctcctcagcctccctggccatcagtgggctccggtccgaggatgaggctgattattactgtgcagcatgggatgacagcctgaatggttatgtcttcggaactgggaccaagctgaccgtccta

SEQ ID NO: 11 - protein, 8 aa, Artificial Sequence
  2447_2H08 HCDR1
GYTFTNYG

SEQ ID NO: 12 - protein, 8 aa, Artificial Sequence
  2447_2H08 HCDR2
ISAYNGNT

SEQ ID NO: 13 - protein, 16 aa, Artificial Sequence
  2447_2H08 HCDR3
DPHILTGYYRGGWFDP

SEQ ID NO: 14 - protein, 6 aa, Artificial Sequence
  2447_2H08 LCDR1
SLRTSY

SEQ ID NO: 15 - protein, 3 aa, Artificial Sequence
  2447_2H08 LCDR2
GKT

SEQ ID NO: 16 - protein, 13 aa, Artificial Sequence
  2447_2H08 LCDR3
HSRDSNDNYLEVV

SEQ ID NO: 17 - protein, 125 aa, Artificial Sequence
  2447_2H08 VH
EVQLVQSGAEVKKPGASVKVSCKASGYTFTNYGISWVRQAPGQGLEWMGWISAYNGNTNYAQKLQGRVTMTTDTSTSTAYMELRSLRSDDTAVYYCARDPHILTGYYRGGWFDPWGQGTTVTVSS

SEQ ID NO: 18 - protein, 110 aa, Artificial Sequence
  2447_2H08 VL
SYELTQDPAVSVALGQTVTITCQGDSLRTSYAGWLQQKPGQAPVLVLYGKTSRPSGIPDRFSGSTSGNTASLTITGAQAEDEAEYFCHSRDSNDNYLEVVFGGGTKLTVL

SEQ ID NO: 19 - protein, 250 aa, Artificial Sequence
  2447_2H08 scFv
EVQLVQSGAEVKKPGASVKVSCKASGYTFTNYGISWVRQAPGQGLEWMGWISAYNGNTNYAQKLQGRVTMTTDTSTSTAYMELRSLRSDDTAVYYCARDPHILTGYYRGGWFDPWGQGTTVTVSSGGGGSGGGGSGGGASSYELTQDPAVSVALGQTVTITCQGDSLRTSYAGWLQQKPGQAPVLVLYGKTSRPSGIPDRFSGSTSGNTASLTITGAQAEDEAEYFCHSRDSNDNYLEVVFGGGTKLTVL

SEQ ID NO: 20 - DNA, 750 bp, Artificial Sequence
  2447_2H08 scFv
gaagtgcagctggtgcagtctggagctgaggtgaagaagcctggggcctcagtgaaggtctcctgcaaggcttctggttacacctttaccaactacggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagatccgcatattttgactggttattataggggagggtggttcgacccctgggggcaagggaccacggtcaccgtctcgagtggtggaggcggttcaggcggaggtggctctggcggtggcgctagctcctatgagctgactcaggaccctgctgtgtctgtggccttgggtcagacagtcaccatcacgtgccaaggagacagcctcagaacctcttatgcaggctggctccagcagaagccaggacaggcccctgtactcgtcctctatggtaaaaccagccggccctcagggatcccagaccgattctctggctccacctcaggaaacacagcttccttgaccatcactggggctcaggcggaggatgaggctgagtatttctgtcactcccgggacagcaatgataactatctagaggtggttttcggcggagggaccaagctgaccgtccta

SEQ ID NO: 21 - protein, 8 aa, Artificial Sequence
  2447_3A01 HCDR1
GFTFDDYA

SEQ ID NO: 22 - protein, 8 aa, Artificial Sequence
  2447_3A01 HCDR2
ISWNSGSI

SEQ ID NO: 23 - protein, 13 aa, Artificial Sequence
  2447_3A01 HCDR3
DISSGWYPGTFDY

SEQ ID NO: 24 - protein, 6 aa, Artificial Sequence
  2447_3A01 LCDR1
SLRSYY

SEQ ID NO: 25 - protein, 3 aa, Artificial Sequence
  2447_3A01 LCDR2
GKN

SEQ ID NO: 26 - protein, 10 aa, Artificial Sequence
  2447_3A01 LCDR3
SSRDSSDDVV

SEQ ID NO: 27 - protein, 122 aa, Artificial Sequence
  2447_3A01 VH
EVQLLESGGGLVQPGGSLRLSCAASGFTFDDYAMHWVRQAPGKGLEWVSGISWNSGSIVYADFVKGRFTISRDNAKNSLYLQMNSLRAEDTALYYCAKDISSGWYPGTFDYWGQGTLVTVSS

SEQ ID NO: 28 - protein, 107 aa, Artificial Sequence
  2447_3A01 VL
SYELTQDPAVSVALGQTVRITCQGDSLRSYYASWYQQKPGQAPVLVIYGKNNRPSGIPDRFSGSSSGNTASLTVTGAQAEDEADYYCSSRDSSDDVVFGGGTQLTVL

SEQ ID NO: 29 - protein, 244 aa, Artificial Sequence
  2447_3A01 scFv
EVQLLESGGGLVQPGGSLRLSCAASGFTFDDYAMHWVRQAPGKGLEWVSGISWNSGSIVYADFVKGRFTISRDNAKNSLYLQMNSLRAEDTALYYCAKDISSGWYPGTFDYWGQGTLVTVSSGGGGSGGGGSGGGASSYELTQDPAVSVALGQTVRITCQGDSLRSYYASWYQQKPGQAPVLVIYGKNNRPSGIPDRFSGSSSGNTASLTVTGAQAEDEADYYCSSRDSSDDVVFGGGTQLTVL

SEQ ID NO: 30 - DNA, 732 bp, Artificial Sequence
  2447_3A01 scFv
gaggtgcagctgttggagtctgggggaggcttggtacagcctggggggtccctgagactctcctgtgcagcctctggattcacctttgatgattatgccatgcactgggtccggcaagctccagggaagggcctggagtgggtctcaggtattagttggaatagtggtagcatagtctatgcggactttgtgaagggccgattcaccatctccagagacaacgccaagaactccctgtatctgcaaatgaacagtctgagagctgaggacacggccttgtattactgtgcaaaagatatttcaagtggctggtacccagggacctttgactactggggccagggaaccctggtcaccgtctcgagtggtggaggcggttcaggcggaggtggctctggcggtggcgctagctcctatgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccgtcactggggctcaggcggaagacgaggctgactattactgtagctcccgggacagcagtgatgatgtggtattcggcggagggacccagctcaccgtccta

SEQ ID NO: 31 - protein, 8 aa, Artificial Sequence
  2447_3A08 HCDR1
GYTFTSYG

SEQ ID NO: 32 - protein, 8 aa, Artificial Sequence
  2447_3A08 HCDR2
ISAYNGNT

SEQ ID NO: 33 - protein, 14 aa, Artificial Sequence
  2447_3A08 HCDR3
DRATIFGVVTPFDY

SEQ ID NO: 34 - protein, 6 aa, Artificial Sequence
  2447_3A08 LCDR1
SIRSYS

SEQ ID NO: 35 - protein, 3 aa, Artificial Sequence
  2447_3A08 LCDR2
GKN

SEQ ID NO: 36 - protein, 11 aa, Artificial Sequence
  2447_3A08 LCDR3
NSRDSSGNRVV

SEQ ID NO: 37 - protein, 123 aa, Artificial Sequence
  2447_3A08 VH
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDRATIFGVVTPFDYWGQGTLVTVSS

SEQ ID NO: 38 - protein, 108 aa, Artificial Sequence
  2447_3A08 VL
SYELTQDPAVSVALGQTVRITCQGDSIRSYSASWYQQKPGQAPRLVIYGKNNRPSGIPDRVSGSTSGNTASLTVTGAQAEDEADYYCNSRDSSGNRVVFGGGTQLTVL

SEQ ID NO: 39 - protein, 246 aa, Artificial Sequence
  2447_3A08 scFv
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDRATIFGVVTPFDYWGQGTLVTVSSGGGGSGGGGSGGGASSYELTQDPAVSVALGQTVRITCQGDSIRSYSASWYQQKPGQAPRLVIYGKNNRPSGIPDRVSGSTSGNTASLTVTGAQAEDEADYYCNSRDSSGNRVVFGGGTQLTVL

SEQ ID NO: 40 - DNA, 738 bp, Artificial Sequence
  2447_3A08 scFv
gaagtgcagctggtgcagtctggagctgaggtgaagaagcctggggcctcagtgaaggtctcctgcaaggcttctggttacacctttaccagctacggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagatcgggctacgatttttggagtggttaccccctttgactactggggccaaggaaccctggtcaccgtctcgagtggtggaggcggttcaggcggaggtggctctggcggtggcgctagctcctatgagctgactcaggaccccgctgtgtctgtggccttgggacagacagtcaggatcacatgccagggagacagtattagaagttattctgccagctggtaccagcagaagccagggcaggcccctcgccttgttatctatggtaaaaacaaccggccctcagggatcccagaccgagtctctggctccacttcaggaaatacagcttccttgaccgtcactggggctcaggcggaagatgaggctgactattactgtaactcccgggacagcagtggtaaccgtgtggtattcggcggagggacccagctcaccgtccta

SEQ ID NO: 41 - protein, 8 aa, Artificial Sequence
  2447_3A09 HCDR1
GFTFDDYA

SEQ ID NO: 42 - protein, 8 aa, Artificial Sequence
  2447_3A09 HCDR2
ISWNSGSI

SEQ ID NO: 43 - protein, 13 aa, Artificial Sequence
  2447_3A09 HCDR3
DVSSGWYWYAFDI

SEQ ID NO: 44 - protein, 6 aa, Artificial Sequence
  2447_3A09 LCDR1
SLRSYY

SEQ ID NO: 45 - protein, 3 aa, Artificial Sequence
  2447_3A09 LCDR2
GKN

SEQ ID NO: 46 - protein, 10 aa, Artificial Sequence
  2447_3A09 LCDR3
NSRDSGGSVV

SEQ ID NO: 47 - protein, 122 aa, Artificial Sequence
  2447_3A09 VH
EVQLLESGGGLVQPGRSLRLSCAATGFTFDDYAMHWVRQAPGKGLEWVSGISWNSGSIGYADSVKGRFTISRDNAKNSLYLQMNSLRAEDTASYYCAKDVSSGWYWYAFDIWGQGTLVTVSS

SEQ ID NO: 48 - protein, 107 aa, Artificial Sequence
  2447_3A09 VL
SYELTQDPAVSVALGQTVRITCQGDSLRSYYASWYQQKPGQAPVLVIYGKNNRPSGIPGRFSGSSSGNTASLTVTGAQAEDEADYYCNSRDSGGSVVFGGGTKLTVL

SEQ ID NO: 49 - protein, 244 aa, Artificial Sequence
  2447_3A09 scFv
EVQLLESGGGLVQPGRSLRLSCAATGFTFDDYAMHWVRQAPGKGLEWVSGISWNSGSIGYADSVKGRFTISRDNAKNSLYLQMNSLRAEDTASYYCAKDVSSGWYWYAFDIWGQGTLVTVSSGGGGSGGGGSGGGASSYELTQDPAVSVALGQTVRITCQGDSLRSYYASWYQQKPGQAPVLVIYGKNNRPSGIPGRFSGSSSGNTASLTVTGAQAEDEADYYCNSRDSGGSVVFGGGTKLTVL

SEQ ID NO: 50 - DNA, 732 bp, Artificial Sequence
  2447_3A09 scFv
gaggtgcagctgttggagtctgggggaggcttggtacagcctggcaggtccctgagactctcctgtgcagccactggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggcctcgtattactgtgcaaaagatgttagcagtggctggtactggtatgcttttgatatctggggccaaggaaccctggtcaccgtctcgagtggtggaggcggttcaggcggaggtggctctggcggtggcgctagctcctatgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccaggccgattctctggctccagctcaggaaacacagcttccttgaccgtcactggggctcaggcggaagatgaggctgactattactgtaattcccgggacagcggtggttctgtggttttcggcggagggaccaagctgaccgtccta

SEQ ID NO: 51 - protein, 8 aa, Artificial Sequence
  2447_3A12 HCDR1
GYTFTSYG

SEQ ID NO: 52 - protein, 8 aa, Artificial Sequence
  2447_3A12 HCDR2
ISAYNGNT

SEQ ID NO: 53 - protein, 16 aa, Artificial Sequence
  2447_3A12 HCDR3
DPHILTGYYRGGWFDP

SEQ ID NO: 54 - protein, 6 aa, Artificial Sequence
  2447_3A12 LCDR1
SLRSYY

SEQ ID NO: 55 - protein, 3 aa, Artificial Sequence
  2447_3A12 LCDR2
GKN

SEQ ID NO: 56 - protein, 11 aa, Artificial Sequence
  2447_3A12 LCDR3
NSRDSSGNHRV

SEQ ID NO: 57 - protein, 125 aa, Artificial Sequence
  2447_3A12 VH
EVQLVQSGAEVKKPGASVKVSCKASGYTFTSYGISWVRQAPGQGLEWMGWISAYNGNTNYAQKLRGRVTMTTDTSTSTAYMELRSLRSDDTAVYYCARDPHILTGYYRGGWFDPWGQGTLVTVSS

SEQ ID NO: 58 - protein, 108 aa, Artificial Sequence
  2447_3A12 VL
SYELTQDPAVSVVLGQTVRITCQGDSLRSYYASWYQQKPGQAPVLVIYGKNNRPSGIPDRFSGSSSGNTASLTITGAQAEDEADYYCNSRDSSGNHRVFGGGTKLTVL

SEQ ID NO: 59 - protein, 248 aa, Artificial Sequence
  2447_3A12 scFv
EVQLVQSGAEVKKPGASVKVSCKASGYTFTSYGISWVRQAPGQGLEWMGWISAYNGNTNYAQKLRGRVTMTTDTSTSTAYMELRSLRSDDTAVYYCARDPHILTGYYRGGWFDPWGQGTLVTVSSGGGGSGGGGSGGGASSYELTQDPAVSVVLGQTVRITCQGDSLRSYYASWYQQKPGQAPVLVIYGKNNRPSGIPDRFSGSSSGNTASLTITGAQAEDEADYYCNSRDSSGNHRVFGGGTKLTVL

SEQ ID NO: 60 - DNA, 744 bp, Artificial Sequence
  2447_3A12 scFv
gaagtgcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccggggcagagtcaccatgaccacagacacatccacgagcacagcctacatggagctgaggagcctgagatctgacgacacggccgtgtattactgtgcgagagatccgcatattttgactggttattataggggagggtggttcgacccctggggccagggcaccctggtcaccgtctcgagtggtggaggcggttcaggcggaggtggctctggcggtggcgctagctcctatgagctgactcaggaccctgctgtgtctgtggtcttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatcgtgtattcggcggagggaccaagctgaccgtccta

SEQ ID NO: 61 - protein, 8 aa, Artificial Sequence
  2447_3B07 HCDR1
GFTFDDYA

SEQ ID NO: 62 - protein, 8 aa, Artificial Sequence
  2447_3B07 HCDR2
TSWDGGST

SEQ ID NO: 63 - protein, 10 aa, Artificial Sequence
  2447_3B07 HCDR3
DHSSGWYNGG

SEQ ID NO: 64 - protein, 6 aa, Artificial Sequence
  2447_3B07 LCDR1
SLRSYY

SEQ ID NO: 65 - protein, 3 aa, Artificial Sequence
  2447_3B07 LCDR2
GKN

SEQ ID NO: 66 - protein, 11 aa, Artificial Sequence
  2447_3B07 LCDR3
NSRDSSGNHVV

SEQ ID NO: 67 - protein, 122 aa, Artificial Sequence
  2447_3B07 VH
QVQLVESGGVVVQPGGSLRLSCAASGFTFDDYAMHWVRQAPGRGLEWVSLTSWDGGSTYYADSVKGRFTISRDNSKNSLYLQMNSLRAEDTALYYCAKDHSSGWYNGGMDVWGQGTMVTVSS

SEQ ID NO: 68 - protein, 108 aa, Artificial Sequence
  2447_3B07 VL
SYELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 69 - protein, 245 aa, Artificial Sequence
  2447_3B07 scFv
QVQLVESGGVVVQPGGSLRLSCAASGFTFDDYAMHWVRQAPGRGLEWVSLTSWDGGSTYYADSVKGRFTISRDNSKNSLYLQMNSLRAEDTALYYCAKDHSSGWYNGGMDVWGQGTMVTVSSGGGGSGGGGSGGGASSYELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 70 - DNA, 735 bp, Artificial Sequence
  2447_3B07 scFv
caggtgcagctggtggagtctgggggagtcgtggtacagcctggggggtccctgagactctcctgtgcagcctctggattcacctttgatgattatgccatgcactgggtccgtcaagctccggggaggggtctggagtgggtctctcttactagttgggatggtggtagcacatactatgcagactctgtgaagggtcgattcaccatctccagagacaacagcaaaaactccctgtatctgcaaatgaacagtctgagagctgaggacaccgccttgtattactgtgcaaaagatcatagcagcggctggtacaacgggggtatggacgtctggggccaagggacaatggtcaccgtctcgagtggtggaggcggttcaggcggaggtggctctggcggtggcgctagctcctatgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatgtggtattcggcggagggaccaagctgaccgtccta

SEQ ID NO: 71 - protein, 8 aa, Artificial Sequence
  2447_3C08 HCDR1
GFTFDDYA

SEQ ID NO: 72 - protein, 8 aa, Artificial Sequence
  2447_3C08 HCDR2
ISWNSGSI

SEQ ID NO: 73 - protein, 15 aa, Artificial Sequence
  2447_3C08 HCDR3
DRGSGYEGNYYGMDV

SEQ ID NO: 74 - protein, 6 aa, Artificial Sequence
  2447_3C08 LCDR1
SLRSYY

SEQ ID NO: 75 - protein, 3 aa, Artificial Sequence
  2447_3C08 LCDR2
GKN

SEQ ID NO: 76 - protein, 11 aa, Artificial Sequence
  2447_3C08 LCDR3
NSRDSSGNHYV

SEQ ID NO: 77 - protein, 124 aa, Artificial Sequence
  2447_3C08 VH
EVQLLESGGGLVQPGGSLRLSCAASGFTFDDYAMHWVRQAPGKGLEWVSGISWNSGSIGYADSVKGRFTISRDNAKNSLYLQMNSLRAEDTALYYCAKDRGSGYEGNYYGMDVWGQGTLVTVSS

SEQ ID NO: 78 - protein, 108 aa, Artificial Sequence
  2447_3C08 VL
SYELTQDPAVSVALGQTVRITCQGDSLRSYYASWYQQKPGQAPVLVIYGKNNRPSGIPDRFSGTTSGNTASLTITGAQAEDEADYYCNSRDSSGNHYVFGTGTKVTVL

SEQ ID NO: 79 - protein, 247 aa, Artificial Sequence
  2447_3C08 scFv
EVQLLESGGGLVQPGGSLRLSCAASGFTFDDYAMHWVRQAPGKGLEWVSGISWNSGSIGYADSVKGRFTISRDNAKNSLYLQMNSLRAEDTALYYCAKDRGSGYEGNYYGMDVWGQGTLVTVSSGGGGSGGGGSGGGASSYELTQDPAVSVALGQTVRITCQGDSLRSYYASWYQQKPGQAPVLVIYGKNNRPSGIPDRFSGTTSGNTASLTITGAQAEDEADYYCNSRDSSGNHYVFGTGTKVTVL

SEQ ID NO: 80 - DNA, 741 bp, Artificial Sequence
  2447_3C08 scFv
gaggtgcagctgttggagtctgggggaggcttggtacagcctggggggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagataggggtagtggctacgaaggaaactactacggtatggacgtctggggccaaggaaccctggtcaccgtctcgagtggtggaggcggttcaggcggaggtggctctggcggtggcgctagctcctatgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggtaccacctcaggaaacacagcctccttgaccatcactggggctcaggcggaagatgaggctgactattactgtaactcccgggacagcagtggtaaccattatgtcttcggaactgggaccaaggtcaccgtccta

SEQ ID NO: 81 - protein, 8 aa, Artificial Sequence
  2447_3H02 HCDR1
GFTFDDYA

SEQ ID NO: 82 - protein, 8 aa, Artificial Sequence
  2447_3H02 HCDR2
ISWDSGSI

SEQ ID NO: 83 - protein, 13 aa, Artificial Sequence
  2447_3H02 HCDR3
DVSSGWYWYAFDI

SEQ ID NO: 84 - protein, 6 aa, Artificial Sequence
  2447_3H02 LCDR1
ILRSYY

SEQ ID NO: 85 - protein, 3 aa, Artificial Sequence
  2447_3H02 LCDR2
GKN

SEQ ID NO: 86 - protein, 11 aa, Artificial Sequence
  2447_3H02 LCDR3
NSRDSSGNRVV

SEQ ID NO: 87 - protein, 122 aa, Artificial Sequence
  2447_3H02 VH
EVQLLESGGGLVQPGRSLRLSCAASGFTFDDYAMHWVRQAPGKGLEWVSGISWDSGSIGYADSVKGRFTISRDNAKNSLYLQMNSLRAEDTALYYCAKDVSSGWYWYAFDIWGQGTLVTVSS

SEQ ID NO: 88 - protein, 108 aa, Artificial Sequence
  2447_3H02 VL
SYELTQDPAVSVALGQTVRITCQGDILRSYYASWYQQKPGQAPVLVIYGKNNRPSGIPDRFSGSSSGNTASLTITGAQAEDEADYYCNSRDSSGNRVVFGGGTKLTVL

SEQ ID NO: 89 - protein, 245 aa, Artificial Sequence
  2447_3H02 scFv
EVQLLESGGGLVQPGRSLRLSCAASGFTFDDYAMHWVRQAPGKGLEWVSGISWDSGSIGYADSVKGRFTISRDNAKNSLYLQMNSLRAEDTALYYCAKDVSSGWYWYAFDIWGQGTLVTVSSGGGGSGGGGSGGGASSYELTQDPAVSVALGQTVRITCQGDILRSYYASWYQQKPGQAPVLVIYGKNNRPSGIPDRFSGSSSGNTASLTITGAQAEDEADYYCNSRDSSGNRVVFGGGTKLTVL

SEQ ID NO: 90 - DNA, 735 bp, Artificial Sequence
  2447_3H02 scFv
gaggtgcagctgttggagtctgggggaggcttggtacagcctggcaggtccctgagactctcctgtgcagcctctggattcacctttgatgattatgccatgcactgggtccggcaagctccagggaagggcctggagtgggtctcaggtattagttgggatagtggtagcataggctatgcggactctgtgaagggccgattcaccatctccagagacaacgccaagaactccctgtatctgcaaatgaacagtctgagagctgaggacacggccttgtattactgtgcaaaagatgttagcagtggctggtactggtatgcttttgatatctggggccagggaaccctggtcaccgtctcgagtggtggaggcggttcaggcggaggtggctctggcggtggcgctagctcctatgagctgactcaggaccctgctgtgtctgtggccttgggacagacagtcaggatcacatgccaaggagacatcctcagaagttattatgcaagttggtaccagcagaagccaggacaggcccctgtacttgtcatttatggtaaaaacaaccggccctcagggatcccagaccgattctctggctccagctcaggaaacacagcttccttgaccatcactggggctcaggcggaagatgaggctgactattactgtaactcccgggacagcagtggtaaccgtgtggtattcggcggagggaccaagctgaccgtccta

SEQ ID NO: 91 - protein, 8 aa, Artificial Sequence
  2447_3H08 HCDR1
GFTFDDYA

SEQ ID NO: 92 - protein, 8 aa, Artificial Sequence
  2447_3H08 HCDR2
ISWNSGSI

SEQ ID NO: 93 - protein, 13 aa, Artificial Sequence
  2447_3H08 HCDR3
DRSSGWYTGSFDY

SEQ ID NO: 94 - protein, 6 aa, Artificial Sequence
  2447_3H08 LCDR1
SLRSYY

SEQ ID NO: 95 - protein, 3 aa, Artificial Sequence
  2447_3H08 LCDR2
GKN

SEQ ID NO: 96 - protein, 11 aa, Artificial Sequence
  2447_3H08 LCDR3
QSRDSSDNRVL

SEQ ID NO: 97 - protein, 122 aa, Artificial Sequence
  2447_3H08 VH
EVQLLESGGGLVQPGRSLRLSCAASGFTFDDYAMHWVRQAPGKGLEWVSGISWNSGSIDYADSVKGRFTISRDNSKNTLYLQMNSLRAEDTAVYYCARDRSSGWYTGSFDYWGQGTLVTVSS

SEQ ID NO: 98 - protein, 108 aa, Artificial Sequence
  2447_3H08 VL
SYELTQDPAVSVALGQTVRITCQGDSLRSYYASWYRQKPGQAPVLVIYGKNNRPSGIPDRFSGSSSGNTASLTIIGAQAEDEADYYCQSRDSSDNRVLFGGGTKVTVL

SEQ ID NO: 99 - protein, 245 aa, Artificial Sequence
  2447_3H08 scFv
EVQLLESGGGLVQPGRSLRLSCAASGFTFDDYAMHWVRQAPGKGLEWVSGISWNSGSIDYADSVKGRFTISRDNSKNTLYLQMNSLRAEDTAVYYCARDRSSGWYTGSFDYWGQGTLVTVSSGGGGSGGGGSGGGASSYELTQDPAVSVALGQTVRITCQGDSLRSYYASWYRQKPGQAPVLVIYGKNNRPSGIPDRFSGSSSGNTASLTIIGAQAEDEADYYCQSRDSSDNRVLFGGGTKVTVL

SEQ ID NO: 100 - DNA, 735 bp, Artificial Sequence
  2447_3H08 scFv
gaggtgcagctgttggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcatagactatgcagactccgtgaagggccgattcaccatctccagagacaattccaagaacacgctgtatctgcaaatgaacagcctgagagctgaggacacagctgtgtactactgtgcgagagatcggagtagtggctggtacacggggtcctttgactactggggccagggcaccctggtcaccgtctcgagtggtggaggcggttcaggcggaggtggctctggcggtggcgctagctcctatgagctgactcaggaccctgctgtgtctgtggccttgggacagacagtcaggatcacatgccaaggagacagcctcagaagctattatgcaagctggtaccggcagaagccaggacaggcccctgtacttgtcatctatggtaaaaacaaccggccctcagggatcccagaccgattctctggctccagctcgggaaacacagcttccttgaccatcattggggctcaggcggaagacgaggctgactattactgtcagtcccgggacagcagtgataaccgtgttctattcggcggagggaccaaggtcaccgtccta

SEQ ID NO: 101 - protein, 8 aa, Artificial Sequence
  G03 HCDR1
GYTFTSYW

SEQ ID NO: 102 - protein, 8 aa, Artificial Sequence
  G03 HCDR2
INPGTGYI

SEQ ID NO: 103 - protein, 7 aa, Artificial Sequence
  G03 HCDR3
STAPFDY

SEQ ID NO: 104 - protein, 6 aa, Artificial Sequence
  G03 LCDR1
QDIKSY

SEQ ID NO: 105 - protein, 3 aa, Artificial Sequence
  G03 LCDR2
YAT

SEQ ID NO: 106 - protein, 9 aa, Artificial Sequence
  G03 LCDR3
LQYDESPYT

SEQ ID NO: 107 - protein, 116 aa, Artificial Sequence
  G03 VH
EVQLVQSGAEVKKPGATVKISCKVSGYTFTSYWMHWVQQAPGKGLEWIGYINPGTGYIEYNQKFKDRVTITADKSTDTAYMELSSLRSEDTAVYYCASSTAPFDYWGQGTLVTVSS

SEQ ID NO: 108 - protein, 107 aa, Artificial Sequence
  G03 VL
EIVLTQSPGTLSLSPGERATLSCKASQDIKSYLSWYQQKPGQAPRLLIYYATRLADGIPDRFSGSGSGTDFTLTISRLEPEDFAVYYCLQYDESPYTFGQGTKLEIK

SEQ ID NO: 109 - protein, 238 aa, Artificial Sequence
  G03 scFv
EVQLVQSGAEVKKPGATVKISCKVSGYTFTSYWMHWVQQAPGKGLEWIGYINPGTGYIEYNQKFKDRVTITADKSTDTAYMELSSLRSEDTAVYYCASSTAPFDYWGQGTLVTVSSGGGGSGGGGSGGGASEIVLTQSPGTLSLSPGERATLSCKASQDIKSYLSWYQQKPGQAPRLLIYYATRLADGIPDRFSGSGSGTDFTLTISRLEPEDFAVYYCLQYDESPYTFGQGTKLEIK

SEQ ID NO: 110 - protein, 113 aa, Artificial Sequence
  mucin 1
SFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSG

SEQ ID NO: 111 - protein, 182 aa, Artificial Sequence
  Synthetic_CD4
TSITAYKSEGESAEFSFPLNLGEESLQGELRWKAEKAPSSQSWITFSLKNQKVSVQKSTSNPKFQLSETLPLTLQIPQVSLQFAGSGNLTLTLDRGILYQEVNLVVMKVTQPDSNTLTCEVMGPTSPKMRLILKQENQEARVSRQEKVIQVQAPEAGVWQCLLSEGEEVKMDSKIQVLSKGL

SEQ ID NO: 112 - DNA, 135 bp, Artificial Sequence
  hinge domain
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 113 - DNA, 81 bp, Artificial Sequence
  CD28 transmembrane domain
ttttgggtgctggtggtggttggtggagtcctggcttgctatagcttgctagtaacagtggcctttattattttctgggtg

SEQ ID NO: 114 - DNA, 123 bp, Artificial Sequence
  CD28 costimulatory domain
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 115 - DNA, 336 bp, Artificial Sequence
  CD3 zeta intracellular signaling domain
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc